(19) United States
(12) Patent Application Publication
(10) Pub. No.: US 2009/0285840 A1
(43) Pub. Date: Nov. 19, 2009

(54) METHODS FOR TREATING PATHOLOGICAL NEOVASCULARIZATION

(75) Inventors: Carl P. Blobel, New York, NY (US); Victor H. Guaquil, Astoria, NY (US); Steven L. Swendeman, Hartdale, NY (US)

Correspondence Address: DECHERT LLP, P.O. BOX 390460, MOUNTAIN VIEW, CA, 94039-0460, US

(73) Assignee: New York Society for the Ruptured and Crippled Maintaining the Hospital for special Surgery, New York, NY (US)

(21) Appl. No.: 12/432,656

(22) Filed: Apr. 29, 2009

Related U.S. Application Data
(60) Provisional application No. 61/048,910, filed on Apr. 29, 2008.

(51) Int. Cl. A61K 39/395; A61K 31/7088; A61K 31/7105; C12Q 1/37; G01N 33/53

(52) U.S. Cl. 424/178.1; 514/44 A; 435/23; 435/7.72

(57) ABSTRACT

Provided herein are compositions and methods that inhibit expression of Adam9 gene products, such as ADAM9 mRNA and/or ADAM9 polypeptides, as a therapeutic approach for the treatment of pathological neovascularization and conditions associated with angiogenesis.

### CROSS REFERENCE TO RELATED APPLICATIONS

This application claims benefit under 35 U.S.C. § 119(e) of application Ser. No. 60/048,910, filed Apr. 29, 2008, the contents of which is incorporated herein by reference.

### STATEMENT REGARDING FEDERALLY SPONSORED RESEARCH

The United States Government may have certain rights in this invention pursuant to support by the National Institutes of Health through Grant Nos. EY15759 and EY12609.

### BACKGROUND

Ocular neovascularization is one of the leading causes of blindness in humans and is found in diverse eye diseases, including diabetic retinopathy, macular degeneration and retinopathy of prematurity (Bradley, et al., 2007, Angiogenesis 10:141-8; Chen and Smith, 2007, Angiogenesis 10:133-40; Friedlander et al., 2007, Angiogenesis 10:89-101). In addition, pathological neovascularization is also believed to have critical roles in other pathologies, such as cancer and rheumatoid arthritis (Khong et al., 2007, Angiogenesis 10:243-58). Although cellular molecules with crucial functions in pathological neovascularization are considered to be important targets for treatment of proliferative retinopathies, cancer and rheumatoid arthritis, the identity of these molecules have not been well delineated, and therapeutics targeting these molecules are lacking. Thus, there is a need to identify these effectors of neovascularization and provide therapeutic approaches based on their identified function.

### SUMMARY

ADAM9, one of the first ADAMs to be identified and characterized, is a membrane-anchored metalloproteinase containing an N-terminal pro-domain, followed by a metalloprotease domain, a disintegrin domain and cysteine-rich region, an EGF-repeat, a transmembrane domain and a cytoplasmic tail with potential SH3-ligand domains. Mice lacking ADAM9 have no evident major abnormalities during development or adult life, but show reduced tumorigenesis in a mouse model for prostate cancer. The description provided herein show that ADAM9 may play a critical role in pathological retinal neovascularization and angiogenesis in tumor cell growth. In view of these findings, in one aspect, the present disclosure provides a method of treating pathological retinal neovascularization by administering to an eye of a patient in need thereof an amount of an ADAM9 inhibitory compound effective to reduce pathological retinal neovascularization. Various eye disorders characterized by pathological neovascularization that may be treatable with ADAM9 inhibitory compounds include, among others, diabetic retinopathy, macular degeneration, and retinopathy of prematurity.

In another aspect, the disclosure provides a method of inhibiting or reducing angiogenesis, comprising administering to a patient in need thereof an amount of an ADAM9 inhibitory compound effective to reduce angiogenesis or neovascularization in said patient.

In another aspect, the disclosure provides a method of treating rheumatoid arthritis, comprising administering to a patient in need of treatment for rheumatoid arthritis an amount of an ADAM9 inhibitory compound effective to reduce neovascularization of a joint of the patient.

Various ADAM9 inhibitory compounds that may be used for the methods, include, among others, an antisense oligonucleotide, a siRNA, a miRNA, a small organic molecule, an enzyme, an antibody, a peptide, a hormone, and a polynucleotide encoding a polypeptide.

A further aspect of the disclosure relates to a method of screening for inhibitors of ADAM9 mediated angiogenesis and/or pathological neovascularization. In some embodiments, the method comprises contacting ADAM9 polypeptide with an ADAM9 target polypeptide in presence of a candidate agent, and determining the presence of proteolyzed target polypeptide, wherein the target polypeptide comprises one or more of polypeptides CD40, EphB4, Flk1, Tie-2, VE-cadherin and VCAM. In some embodiments, the ADAM9 target polypeptides comprise at least EphB4. In some embodiments, the target polypeptides, in addition to one or more of the foregoing, includes one or more of FGFR2iiib and EGF.

The screening can be carried out in-vitro by contacting ADAM9 polypeptide and target polypeptide in presence of a candidate inhibitor compound, and detecting the proteolyzed products of the target polypeptide. In some embodiments, the screening can be a cell-based system where a cell is incubated with a candidate inhibitor compound and ADAM9 polypeptide and one or more of target polypeptides are co-expressed in the cell. Which ever system is used, in some embodiments, the target polypeptide can be bound to a detectable moiety, such as a fluorescent molecule, a reporter enzyme, a fluorescent protein, for detecting the proteolyzed target polypeptide.

Virtually any candidate compound can be screened, including among others, an antisense oligonucleotide, a siRNA, a miRNA, a small organic molecule, an enzyme, an antibody, a peptide, a hormone, and a polynucleotide encoding a polypeptide.

### BRIEF DESCRIPTION OF THE FIGURES

FIG. 1A and 1B shows quantitation of vascular cell nuclei that have crossed the internal limiting membrane and entered the vitreous side of the retina in sections of retinas of Adam9 −/− or wild type mice that have been subjected to the oxygen induced retinopathy (OIR) model (see Examples). Comparison of neovascular nuclei in Adam9 −/− and wildtype littermates from matings of Adam9 +/− parents are given in FIG. 1A while comparison of neovascular nuclei of whole litters of F2 Adam9 −/− or wildtype mice that were derived from the same FO Adam9 +/− grandparents are given in FIG. 1B. FIG. 1C shows a representative whole mount section of a retina from an Adam9 −/− mouse or wildtype control that was subjected to the OIR model. FIG. 1D shows quantification of the central avascular area that develops during the hyperoxic phase of the OIR model in Adam9 −/− mice and wildtype controls. The inset provides an example for how the avascular area was measured: The area of the whole mount was outlined in green, and the avascular area was outlined in red using Adobe Photoshop®, the size of the areas were determined with NIH Imagequant software, and used to calculate the ratio of these two areas (as described in Examples section).

FIG. 2A-2C shows immunofluorescence and immunohistochemical analysis of sections or whole mounts of retinas from Adam9 −/− or wildtype mice following the OIR model. Whole mounts of retinas from Adam9 −/− or wildtype mice subjected to the OIR models were incubated with a monoclonal antibody against ADAM9 (green), or an antibody against the pericyte marker NG2 (texas red labeled) (FIG. 2A), or with lectin FITC (green) to detect endothelial cells and NG2 (texas red) (FIG. 2B), or with lectin TRITC (red) and an anti-ADAM9 monoclonal antibody (green) (FIG. 2C). Both ADAM9 and NG2 are detected in pathological neovascular tufts, but the staining does not overlap, whereas the staining of ADAM9 overlaps with that of lectin TRITC, suggesting that ADAM9 is upregulated in endothelial cells and not in pericytes. FIG. 2D shows histochemical and immunofluorescent staining of sections of retinas of Adam9 −/− or wildtype controls using monoclonal antibodies against ADAM9. FIG. 2E shows western blot analysis of ADAM9 in retinas from wildtype mice between 12 and 16 days post partum, where 12 days (second lane from left) corresponds to the time the animals were removed from the high oxygen chamber and placed in room air, and day 16 (6th lane from left) represents the endpoint of the OIR experiment. No major differences in the global expression of ADAM9 are detected in retinal extracts, despite the high local upregulation of ADAM9 in pathological neovascular tufts.

FIG. 3A and 3B show growth characteristics of heterotopically injected tumor cells in Adam9 −/− or wild type mice. Litters of age matched Adam9 −/− mice or wildtype controls derived from the same Adam9 +/− grandparents were injected with 10⁶ B16F0 melanoma cells. Each experiment was terminated when the first tumors began to reach a size of larger than 1 cm in diameter. The relative weights of the tumors removed from Adam9 −/− or wildtype mice are shown in FIG. 3A (the average weight of tumors in wild type mice in a given experiment was set to 1, and used to calculate the relative weights of tumors in compared to the average weight of wild type tumors in three separate experiments), and sections of representative tumors to evaluate the pattern, spacing and size of the tumor vasculature is shown in FIG. 3B.

FIG. 4 shows results of experiments on shedding of membrane receptors with known roles in angiogenesis by ADAM9 in “gain of function” overexpression experiments. ADAM9 was coexpressed with alkaline phosphatase tagged fusion proteins of several receptors with critical roles in angiogenesis in Cos-7 cells. Co-expression of the catalytically inactive ADAM9 E>A served as a control, as did expression of the AP-fusion protein alone. The panels represent the amount of alkaline phosphatase activity released into the supernatants under various conditions (see Examples). Overexpression of ADAM9 led to increased release of the ectodomain of Tie-2, VE-cadherin, FLK1, Ephrin B4, CD40 and VCAM, but not of Ephrin B2, E-selectin or ICAM.

FIG. 5 provides results showing that reactive oxygen species stimulate the activity of endogenous ADAM9 in mouse embryonic fibroblasts. To assess the regulation of endogenous ADAM9, mouse embryonic fibroblasts from Adam9 −/− mice or wildtype controls were transfected with alkaline phosphatase-tagged Ephrin B4 (EphB4) to monitor its shedding in the presence of various concentrations of H₂O₂ for 6 hrs (0-200 μM). Increased shedding of EphB4 after treatment with H₂O₂ was only seen in wildtype cells (left panel), but not in Adam9 −/− cells (right panel).

FIG. 6 shows the amino acid sequence of human ADAM9 (SEQ ID NO: 1);

FIG. 7 shows the amino acid sequence of isoform 1 precursor of human ADAM9 (SEQ ID NO:2);

FIG. 8 shows the amino acid sequence of isoform 2 precursor of human ADAM9 (SEQ ID NO:3);

FIG. 9 shows the nucleotide sequence of variant 2 mRNA (SEQ ID NO:4) encoding human ADAM9 polypeptide;

FIG. 10 shows the nucleotide sequence of variant 1 mRNA (SEQ ID NO:5) encoding human ADAM9 polypeptide;

FIG. 11 shows the ADAM9 nucleotide sequence used for generating antisense oligonucleotides in Table 1 (SEQ ID NO:94).

### DETAILED DESCRIPTION

The various inventions described herein are based upon the finding that one member of the family of metalloprotease disintegrins referred to as ADAMs, a disintegrin and metalloprotease 9 (“ADAM9”), also referred to as MDC9 or Meltrin γ, is an important mediator of pathological neovascularization.

The ADAMs are a family of almost 30 known multidomain proteins so named because they contain a disintegrin and metalloprotease domain. ADAMs share high sequence homology and domain organization with snake venom metalloproteases; both contain a metalloprotease-like domain with an associated regulatory prodomain, a disintegrin-like domain, a cysteine-rich domain, and an epidermal growth factor-like domain. In addition, ADAMs contain a transmembrane domain and a cytoplasmic tail (Evans, 2001, BioEssays 23:628-639). The metalloprotease domains are involved in cleavage of the amyloid precursor protein and ectodomain shedding of several membrane-anchored proteins, including tumor necrosis factors. The disintegrin and cysteine-rich domains play important roles in cell-cell and cell-matrix adhesion, cell differentiation, and fusion (Primakoff and Myles, 2000, Trends Genet 16:83-87). The cytoplasmic tails contain consensus SH3-binding motifs, and have been shown to bind p85, Src, and alpha-actinin-1 and -2, suggesting that the cytoplasmic tail region is involved in signal transduction (Blobel, C. P., 1997, Cell, 90:589-592; Blobel, C. P., 2005, Nat Rev Mol Cell Biol. 6(1):32-43; Cao et al., 2001, Biochem. J. 357:353-361; Galliano et al., 2000, J. Biol. Chem. 275:13933-13939; Kang et al., 2001, J. Biol. Chem. 276:24466-24472; Kang et al., 2000, Biochem. J. 352:883-892; and Suzuki et al., 2000, Oncogene, 19:5842-5850).

ADAM-9 expression has been found in rat kidney and also been shown to be up-regulated in hepatocellular carcinomas using protein microarrays. However, the significance of this finding to cancer progression is unclear. ADAM-9 has also been shown to be involved in the shedding of heparin-binding epidermal growth factor (HB-EGF) in the processing of the amyloid precursor protein, and as a ligand for several integrins. Although it is highly conserved between mouse, human and Xenopus and it is widely expressed in different tissues, a knock-out mouse lacking ADAM-9 shows no evident pathological abnormalities. Polyclonal and monoclonal antibodies against ADAM-9 have been used in the identification of novel proteins associated with hepatocellular carcinoma in protein arrays (Tannapfel et al., 2003, J Pathology 201:238-49) and also in atherosclerosis models using venous vascular smooth muscle cells (VSMCs) to assay for ADAM9 expression (Al-Fakhri et al., 2003, J Cell. Biochem. 89:808-823). However, the significance of ADAM9 as a marker is unclear. ADAM9 shows heightened expression at the blastocyst implantation site, suggesting its involvement in implantation. ADAM9 has also been found to be highly expressed in epithelial cells of animal model systems for prostate, breast, and gastrointestinal carcinoma. Studies on ADAM9 deficient animals suggest that it affects tumor progression because there is reduced tumorigenesis in mouse model for prostate cancer in animals lacking ADAM9 (Peduto et al., 2005, Cancer Research 65:9312-9).

However, there has been no previous indication that ADAM9 is involved in pathologies related to retinal neovascularization or that ADAM9 may affect angiogenesis. The studies herein used a mouse model of retinopathy of prematurity (ROP), the oxygen induced retinopathy (OIR) model, in which animals are subjected to high oxygen levels. This model allows an evaluation of the role of a specific molecule, e.g., gene product, in proliferative retinopathies. Without being bound by theory, in the OIR model, relative hypoxia following the exposure of the animal to high concentrations of oxygen (75%) and return to room air (e.g., 21% oxygen) stimulates production of a hypoxia inducible factor and then VEGF in order to attract new blood vessels. This results in proliferation of vascular cells in the retina and inappropriate formation of pathological vascular tufts, potentially leading to blindness. For example, ROP is a leading cause of blindness in infants in developed and middle income countries due to exposure of preterm neonates to high levels of oxygen. The studies herein provide direct evidence that ADAM9 plays a critical role in pathological neovascularization in the mouse OIR model. Specifically, the strong decrease in pathological retinal neovascularization in Adam9 −/− mice compared to wildtype animals demonstrates that ADAM9 is required for the development pathological vessels in the retina, even though lack of ADAM9 does not lead to evident defects in angiogenesis during development (Weskamp et al., 2002, Mol Cell Biol. 22(5):1537-1544). In ADAM9 −/− null animals, there is a significant reduction in number of vascular cells that contribute to pathological neovascularization. These results are consistent with the observation that ADAM9 is highly expressed in the pathological vascular tufts that develop after mice are subjected to the OIR model, but are not seen during the normal postnatal development of the mouse retinal vasculature. The expression of ADAM9 in neovascular tufts was found in cells that were also labeled with the endothelial cell marker FITC lectin, but did not overlap with expression of NG2, a marker for pericytes. Moreover, it is found that cells positive for the pericyte marker NG2 and cells expressing ADAM9 closely intermingled in neovascular tufts, whereas pericytes are usually found ensheathing and surrounding normal vessels and capillaries.

Following exposure to oxygen in the OIR model, mice lacking ADAM9 also display a significantly enlarged central avascular area in the retina compared to wildtype controls. When retina from Adam9 −/− mice or wild type controls were compared immediately after mice were exposed to high concentrations of oxygen for 5 days, the size of the central avascular area was very similar. These results demonstrate that the vascular regression caused by exposure to high oxygen levels was not detectably affected in the absence of ADAM9. However, once neovascularization was initiated through relative hypoxia, ADAM9 appears to be important for the generation of new capillaries that grow into the central avascular area, further supporting the conclusion that ADAM9 is important for neovascularization in the retina.

Another approach used for evaluating pathological neovascularization is determining the growth characteristics of heterotopically injected tumor cells in mice. Angiogenesis may be critical for tumor establishment and growth through the recruitment and growth of blood vessels into the tumor growth site. Injection of B16F0 melanoma cells into Adam9 −/− and wildtype mice showed that tumor growth was strongly reduced in the ADAM9 −/− animals. This observation on tumor growth and the reduced OIR neovascularization suggested that ADAM9 cells may have important functions in endothelial cell growth and angiogenesis. ADAM9 could affect angiogenesis by processing membrane proteins that regulate angiogenesis and neovascularization. To address this possibility, an assay was developed using Cos-7 cells that express ADAM9 as well as membrane-anchored receptors known to have critical functions in angiogenesis. These included CD40, EprinB2, EphB4, E-selectin, Flk-1, ICAM-1, VCAM, Tie-2, VE-Cadherin, CD34 and CD36. In coexpression experiments with ADAM9 and the candidate substrates, overexpression of ADAM9 increased the release of CD40, EphB4, Flk-1, Tie-2, VE-cadherin and VCAM compared to cells expressing an inactive form of ADAM9 (E>A catalytic site mutation). Overexpression of ADAM9 did not increase the shedding of E-selectin, ICAM-1, Ephrin B2, CD34 and CD36 compared to ADAM9 E>A under the assay conditions. Moreover, when mouse endothelial cells are exposed to H₂O₂, which mimics reactive oxygen species (ROI), there was an increase in expression of ADAM9 as well as release of proteolyzed EphB4, when the EphB4 was transiently expressed in the H₂O₂ treated cells. Cells from ADAM9 −/− subjected to treatment with H₂O₂ did not show an increase in release of proteolyzed EphB4

Taken together, these results are consistent with a role of ADAM9 in the formation of the pathological neovascularization as well as in angiogenesis, although a requirement for ADAM9 in the context of other host-derived contributions to tumor growth, such as release of growth factors from fibroblasts or macrophages, cannot be ruled out.

In accordance with the above, in some embodiments, ADAM9 inhibitory compounds described herein can be used in a method of treating pathological retinal neovascularization, comprising administering to an eye of a patient in need of treatment for pathological retinal neovascularization an amount of an ADAM9 inhibitory compound effective to reduce pathological retinal neovascularization.

Pathological retinal neovascularization is found in a variety of eye diseases, including, among others, diabetic retinopathy, macular degeneration, and retinopathy of prematurity (ROP). Accordingly, in some embodiments, the pathological retinal neovascularization in need of treatment is associated with diabetic retinopathy. In diabetic retinopathy, neovascularization occurs outside the retina and along the surface of the vitreous or into the vitreous cavity when the retina itself is not receiving sufficient oxygen because of diabetes associated damage to blood vessels. The increased vascularization may result in among others, tissue damage and vitreous hemorrhage, which can result in retinal detachment and subsequent blindness.

In some embodiments, the pathological retinal neovascularization in need of treatment is associated with macular degeneration, particularly wet macular degeneration. Neovascularization in macular degeneration generally occurs beneath the retina and macula and may result in hemorrhage and fluid leakage, causing damage to the macula and cell death. The degeneration of the macula leads to loss of central vision.

In some embodiments, the pathological retinal neovascularization in need of treatment is associated with retinopathy of prematurity (ROP). This form of pathological neovascularization can occur when preterm neonates are exposed to high levels of supplemental oxygen or chronic hyperoxia, although other risk factors are associated with the condition. Regardless of the actual trigger, the normal growth of blood vessels in the retina of premature infants appears to be attenuated such that areas without adequate blood supply may initiate angiogenesis to cause growth of abnormal blood vessels. As with other types of pathological retinal neovascularization, these abnormal growths may lead to hemorrhaging and scarring, and deformation of the macula and retinal detachment.

In some embodiments, the ADAM9 inhibitory compounds can be used in a method of treating rheumatoid arthritis, where the method comprises administering to a patient in need of treatment for rheumatoid arthritis an amount of an ADAM9 inhibitory compound effective to reduce neovascularization of a joint of the patient. Pathological neovascularization can occur in the joints through the proliferation of synovial macrophages and fibroblasts after a triggering incident, possibly autoimmune or infectious. Lymphocytes infiltrate perivascular regions along with endothelial cell proliferation, resulting in neovascularization in the affected joint. These blood vessels can become occluded with small clots or inflammatory cells, leading to damage of cartilage and bone.

As noted above, establishment and growth of tumors are affected by the ability to induce new blood vessel growth to supply nutrients to the tumor. Tumor cells are known to produce factors, such as VEGF, that can induce angiogenesis at the tumor site. In some embodiments, the ADAM9 inhibitory compounds can be used in a method of inhibiting or reducing angiogenesis, where the method comprises administering to a patient in need thereof an amount of an ADAM9 inhibitory compound effective to reduce angiogenesis in the patient. Inhibiting angiogenesis may be appropriate for treatment of various cancers, particularly cancers in which ADAM9 is increased and is characterized by angiogenesis, such as prostate cancer, non-small cell lung cancer, pancreatic ductal adenocarcinoma, renal cell carcinoma, prostate, and breast cancer.

The methods above can be used to treat a subject afflicted with the condition, e.g., therapeutic treatment, or used prophylactically to prevent or reduce the occurrence of the condition. Subjects who have not developed the condition but who may be predisposed to developing the condition may be treated prophylactically. “Therapeutic treatment” is a treatment administered to a subject who displays symptoms or signs of pathology, disease, or disorder, in which treatment is administered to such subject for the purpose of diminishing, eliminating or ameliorating those signs or symptoms of pathology, disease, or disorder. “Ameliorating” or “ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation.

“Prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as a nucleic acid, vector, gene, polypeptide, protein, substance, or composition thereof that, when administered to a subject who does not display signs or symptoms of pathology, disease or disorder, or who displays only early signs or symptoms of pathology, disease, or disorder, diminishes, prevents, or decreases the risk of the subject developing a pathology, disease, or disorder.

The various methods and compositions described herein utilize ADAM9 inhibitory compounds. As used herein, an “ADAM9 inhibitory compound” is a compound that inhibits an activity of an ADAM9 polypeptide, or expression of an Adam9 gene product, such as for example synthesis of mRNA encoding an ADAM9 polypeptide (transcription) and/or synthesis of an ADAM9 polypeptide from ADAM9 mRNA (translation).

Compounds that inhibit an activity of an ADAM9 polypeptide can inhibit the protease activity of the metalloprotease domain of ADAM9. The chemical nature of the compounds can vary, and can range from small organic molecules, to small biological molecules such as peptides, hormones, oligonucleotides, to large biological molecules such as polypeptides, enzymes, antibodies and polynucleotides. It will be appreciated that this list is merely illustrative of representative types of ADAM9 inhibitory compounds that can be employed in the methods and compositions described herein, and is not intended to be limiting.

In some embodiments, the ADAM9 inhibitory compound inhibits the protease activity of the metalloprotease domain of an ADAM9 polypeptide. Such ADAM9 inhibitory compounds are known in the art, and include by way of example and not limitation, the class of inhibitors called “hydroxamate inhibitors,” which specifically intersect in a bidentate manner via the hydroxyl and carbonyl oxygens of their hydroxamic group with the zinc ion in the catalytic site (Armour et al., 2002, FEBS Lett. 524(1-3):154-8; Koike et al., 1999, Biochem J. 343:371-5; Bode et al., 1994, EMBO J. 13:1263-1269).

Such hydroxymate inhibitors are typically composed of a carbon back-bone (See, e.g., WO 95/29892, WO 97/24117, WO 97/49679 and EP 0780386), a peptidyl back-bone (WO 9005719, WO 93/20047, WO 95/09841 and WO 96/06074) a peptidomimetic back-bone (Schwartz et al., 1992, Progr. Med. Chem. 29: 271-334; Rasmussen et al., 1997, Pharmacol. Ther. 75:69-75; Denis et al., 1997, Invest. New Drugs 15:175-185). Alternatively, they contain a sulfonamide sulfonyl group which is bonded on one side to a phenyl ring and a sulfonamide nitrogen which is bonded to an hydroxamate group via a chain of one to four carbon atoms (See, e.g., EP 0757984).

Other peptide-based matrix metalloprotease inhibitors that can be used as ADAM9 inhibitory compounds in the methods described herein include thiol amides which exhibit collagenase inhibition activity (U.S. Pat. No. 4,595,700), N-carboxyalkyl derivatives containing a biphenylethylglycine which inhibit MMP-3, MMP-2 and collagenase (Durette, et al., WO 95/29689), lactam derivatives which inhibit matrix metalloproteases, TNF-α and aggrecanase (see U.S. Pat. No. 6,495,699), tricyclic sulfonamide compounds (see U.S. Pat. No. 6,492,422), the compound ONO-4817 (Ono Pharmaceutical Co. Ltd., Osaka, Japan; see also Mori, et al., 2002, Anticancer Res., 22(6C):3985-8) and the collagenase inhibitors GM6001 (trade name Galardin) and GM1489 (a derivative of GM6001) (see U.S. Pat. No. 6,759,432).

Antibodies capable of inhibiting the activity of matrix metalloproteases, and which are expected to be useful as ADAM9 inhibitory compounds in the compositions and methods described herein, are also known in the art. For example, one class of natural inhibitors is monoclonal antibodies. Antibodies which may inhibit the activity of ADAM9 are described in US 2006/0172350, incorporated herein by reference. Similarly, skilled artisans can raise antibodies against polypeptide sequences within the catalytic domain of ADAM9 to be used as ADAM9 inhibitory compounds.

In some embodiments, the antibody can be in the form of an antibody fragment, an Fab fragment, an Fab′ fragment, an F(ab′)2 fragment, an Fv fragment, a linear antibody, a humanized antibody, a monoclonal antibody, a chimeric antibody, a single chain antibody, a diabody, an aptamer and an isolated complementarity determining region fused to another molecule, wherein the ADAM9 inhibitory compound has binding specificity for ADAM9 protein. In some embodiments, the inhibitory ADAM9 antibodies are humanized antibodies comprising an antigen-binding site derived from a non-human immunoglobulin. Method for making such antibodies have been described, including antibodies having their associated non-human V-regions fused to human constant domains (Winter and Milstein, 1991, Nature 349:293-299; Lobuglio et al., 1989, Proc. Nat. Acad. Sci. USA 86:4220-4224; Shaw et al., 1987, J Immunol. 138:4534-4538 ; and Brown et al., 1987, Cancer Res. 47:3577-3583), non-human CDRs grafted into a human supporting FR prior to fusion with an appropriate human antibody constant domain (Riechmann et al., 1988, Nature 332:323-327; Verhoeyen et al., 1988, Science 239:1534-1536; and Jones et al., 1986, Nature 321:522-525), and rodent CDRs supported by recombinantly veneered rodent FRs (European Patent Publication No. 0519596). These “humanized” molecules are designed to minimize unwanted immunological response toward non-human antihuman antibody molecules, which limits the duration and effectiveness of therapeutic applications of those moieties in human recipients.

In some embodiments, the antibodies or binding fragments thereof, are capable of inhibiting the ADAM9 metalloproteinase activity. The metalloproteinase domain of ADAM9 extends from amino acid residue of about 206 to about 413 of the human ADAM9 amino acid sequence. All of the metalloprotease region or various peptides thereof can be used for the preparation of antibodies that are capable of inhibiting the metalloprotease activity of ADAM9. In some embodiments, the antibodies bind to the metalloprotease consensus amino acid sequence of HELGHNLGMNHD (amino acid residues 347-358) (SEQ ID NO:93). In some embodiments, the antibodies bind to an amino acid sequence flanking the metalloprotease consensus sequence region, such as a peptide of 10-20 amino acids on the amino terminal side or of 10-20 amino acids on the carboxy terminal side of the metalloprotease consensus sequence. In some embodiments, function-blocking antibodies against any part of the extracellular domain of ADAM9 can be identified in as inhibitors of its metalloprotase activity in cell based assays.

In some embodiments, the antibodies or binding fragments thereof, are capable of inhibiting the ADAM9 disintegrin activity. The disintegrin domain of ADAM9 extends from amino acid residues of about 414 to about 503 of the human ADAM9 amino acid sequence. All of the disintegrin region or various peptides thereof can be used for the preparation of antibodies that are capable of inhibiting the disintegrin activity of ADAM9.

As further described herein, the antibody or fragment thereof, can be conjugated to a moiety, such as, for example, a toxin (e.g., diphtheria toxin), a radioactive isotope, a drug (e.g., a chemotherapeutic agent), or a fluorochrome.

As will be recognized by skilled artisans, matrix metalloprotease inhibitors such as those described above may be “promiscuous” or “non-selective,” in that they may inhibit more than one matrix metalloprotease. While such promiscuous or non-selective inhibitors having requisite activity against ADAM9 are expected to be useful in the compositions and methods described herein, in some embodiments, compounds that specifically or selectively inhibit the activity of ADAM9 are preferred. Compounds can be tested for any desired degree of specificity or selectively for ADAM9 using routine screening assays.

In some embodiments, the ADAM9 inhibitory compound is a compound that inhibits expression of an Adam9 gene product. Such ADAM9 inhibitory compounds can inhibit Adam9 gene expression by inhibiting the transcription of the Adam9 gene (i.e., by inhibiting synthesis of ADAM9 mRNA) and/or by inhibiting the translation of ADAM9 mRNA (i.e., by inhibiting synthesis of ADAM9 polypeptides). In some embodiments, such ADAM9 inhibitory compounds are oligonucleotides that are complementary to, or that specifically hybridize with, one or more nucleic acids encoding ADAM9, such as the Adam9 gene or ADAM9 mRNA. The hybridization of the oligonucleotide with the nucleic acid encoding ADAM9 interferes with the normal function of the encoding nucleic acid.

As used herein, “hybridization” means hydrogen bonding, which may be Watson-Crick, Hoogsteen or reversed Hoogsteen hydrogen bonding, between complementary nucleoside or nucleotide bases (nucleobases). For example, adenine and thymine are complementary nucleobases which pair through the formation of Watson-Crick hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise base pairing such that stable and specific binding occurs between the oligonucleotide and the DNA or RNA. It is understood in the art that the sequence of an oligonucleotide need not be 100% complementary to its target nucleic acid to be specifically hybridizable.

An antisense compound is specifically hybridizable when binding of the compound to a target DNA or RNA molecule interferes with the normal function of the target DNA or RNA to cause a loss of activity, and there is a sufficient degree of complementarity to avoid non-specific binding of the oligonucleotide compound to non-target sequences under conditions in which specific binding is desired, i.e., under physiological conditions in the case of in vivo assays or therapeutic treatment, and in the case of in vitro assays, under conditions in which the assays are performed.

Oligonucleotide compounds capable of specifically hybridizing to nucleic acids encoding polypeptides are well-known in the art, and include by way of example, and not limitation, antisense oligonucleotides, small interfering RNAs (siRNAs), micro RNA (miRNA), short hairpin RNAs (shRNAs) and antisense expression vectors. Antisense oligonucleotides, siRNA and miRNA capable of inhibiting expression of Adam9 gene products can be designed from the sequences of polynucleotides encoding ADAM9, such as the Adam9 gene and ADAM9 mRNA sequences, using principles that are well known in the art. For example, antisense ADAM9 inhibitory compounds can be designed from polynucleotide sequences encoding ADAM9 as described in US 2004/0092466 (represented as SEQ ID NO:94 herein); Genbank Accession No. NM_{—}003816.2; and Genbank Accession No. NM_{—}001005845.1. The genomic sequence for description of intron sequences are provided in Genbank Accession No. NC_{—}000008.9 and NT_{—}007995.14. siRNA ADAM9 inhibitory compounds can be designed from ADAM9 encoding polynucleotides following the descriptions in Cao et al., 2003, Mol. Cell Biol. 123(19):6725-6738 for related metalloproteases and WO2008154482 for other angiogenic molecules. miRNA ADAM9 inhibitory compounds can be designed from ADAM9 encoding polynucleotides as described on several commercial websites (see, for example, world wide web at dharmacon.com). As will be recognized by skilled artisans, the lengths of such antisense, siRNA and miRNA molecules can vary, as can their degree of complementarity to the ADAM9 encoding polynucleotide. In some embodiments, antisense oligonucleotide ADAM9 inhibitory compounds will contain from 8-80, 8-70, 8-60, 8-50, 8-40, 8-35, 8-30, 8-25, 8-20, 8-15 or 8-10 nucleotides; siRNA ADAM9 will contain from 20 to 22 nucleotides; and miRNA ADAM9 inhibitory compounds will typically contain from 21 to 24 nucleotides. Oligonucleotide ADAM9 inhibitory compounds, including antisense, siRNA and miRNA oligonucleotides, need not exhibit 100% complementarity to the target region of the ADAM9-encoding nucleic acid to be effective in the methods and compositions described herein. Depending upon their length, oligonucleotides exhibiting between 80-100% complimentarity are expected to be effective, although in some circumstances oligonucleotides having even lower percentages of complementarity will be effective. The only requirement is that the oligonucleotide be capable of specifically hybridizing to its target region of the ADAM9-encoding nucleic acid. The correlation between the length of an oligonucleotide, its degree or percentage of complementary to a target nucleic acid sequence and its ability to specifically hybridize to the target sequence under various different hybridization conditions is well understood (see, e.g., Nucleic Acid Hybridization: A Practical Approach, Haines & Higgins, Eds., IRL Press, Oxford, 1988). Oligonucleotide ADAM9 inhibitory compounds having the requisite degree of complementarity as a function of length to be useful in the methods and compositions described herein can be designed using these well-known principles.

In some embodiments, the oligonucleotide ADAM9 inhibitory compound is 90-100% complementary to a target region of ADAM9-encoding nucleic acid, with a degree of complementarity of greater than 95, 96, 97, 98 or 99% being preferred. In some embodiments the oligonucleotide ADAM9 inhibitory compound is 100% complementary (“completely complementary”) to a target region of an ADAM9-encoding nucleic acid. Percentage of complementarity of an oligonucleotide to a region of a target nucleic acid can be determined routinely using basic sequence alignment search tools (BLAST programs; see, e.g., Altschul et al., 1990, J. Mol. Biol. 215:403-410; Zhang & Madden, 1997, Genome Res. 7:649-656).

The target region may be located in the untranslated region of the ADAM9-encoding nucleic acid or in the coding region, or may span the untranslated and coding regions. The targeted region can include the nucleic acid sequence surrounding the AUG start codon, which can include the AUG codon itself, sequences containing and/or surrounding the splice donor and/or acceptor sites in the introns of the Adam9 encoding unprocessed mRNA transcript; sequences containing and/or surrounding the splice donor and/or acceptor sites in the exons of the Adam9 unprocessed mRNA transcript; sequences spanning the intro-exon junctions of the unprocessed Adam9 mRNA transcript; the 3′ untranslated regions of the Adam9 mRNA, and splicing regulator sites in the exons generally located on the 5′ portion of the exon. In some embodiments, an Adam9 specific antisense oligonucleotide can target the splicing acceptor or donor regions to cause exon skipping, thereby inhibiting expression of the ADAM9 polypeptide. These approaches for inhibiting expression are described in, among others, U.S. Pat. No. 6,784,291; U.S. Pat. No. 6,210,892; U.S. Pat. No. 6,653,466; and U.S. patent publication 2002/0055481; all publications incorporated herein by reference.

A variety of different antisense oligonucleotides that can be used as ADAM9 inhibitory compounds in the methods and compositions described herein are also described in US 2004/0092466, the disclosure of which is incorporated herein by reference. Specific antisense oligonucleotides contemplated to be useful in the methods and compositions described herein are provided in TABLE 1 of US 2004/0092466, identified herein as SEQ ID NOS:23-59, respectively, based on the ADAM9 sequence of SEQ ID NO:94, which is disclosed as SEQ ID NO:4 in US 2004/0092466. The nucleotide sequences of these oligonucleotides, as well as the specific antisense oligonucleotides containing these sequences described in Example 15 of US 2004/0092466, are incorporated herein by reference. The nucleotide sequences of these antisense oligonucleotides are reproduced below:

TABLE 1

| | | | | | |
|---|---|---|---|---|---|
| | TARGET | | | | SEQ |
| | SEQ ID | TARGET | | % | ID |
| REGION | NO | SITE | SEQUENCE | INHIB | NO |

| | | | | | |
|---|---|---|---|---|---|
| Coding | 94 | 633 | cctcttcatcctttgcagtt | 84 | 23 |
| Coding | 94 | 866 | gtttccattggtccaaatct | 77 | 24 |
| Coding | 94 | 1119 | caagattatgacccaattca | 69 | 25 |
| Coding | 94 | 1275 | gaatattaagaaggcagttt | 27 | 26 |
| 3′UTR | 94 | 3357 | ctgctttattatagccatga | 85 | 27 |
| 3′UTR | 94 | 2769 | atgactgaataagacttatt | 68 | 28 |
| 3′UTR | 94 | 2910 | tacattaatgataactaagg | 68 | 29 |
| 3′UTR | 94 | 3375 | aagattttataattgctcct | 71 | 30 |
| Coding | 94 | 550 | cgataaatgatgtgctcaaa | 36 | 31 |
| Coding | 94 | 1071 | cagtgatttgtccaaacaca | 30 | 32 |
| Coding | 94 | 1127 | attcattccaagattatgac | 75 | 33 |
| 3′UTR | 94 | 3454 | ttaatagcaagctttgaaag | 68 | 34 |
| Coding | 94 | 693 | gggtctgtggcaagacagct | 65 | 35 |
| Coding | 94 | 1164 | tctttgctccacaggaacaa | 77 | 36 |
| Coding | 94 | 444 | caatggatgaattatgaact | 62 | 37 |
| Coding | 94 | 2096 | tccgtatcctttagtctcac | 80 | 38 |
| 3′UTR | 94 | 3550 | ttccttctttctccagccta | 81 | 39 |
| Coding | 94 | 2515 | gtgagggaactatataaagg | 66 | 40 |
| Coding | 94 | 2474 | aggaattaagtttccctgag | 74 | 41 |
| 3′UTR | 94 | 3667 | aaaattttacgatccatacc | 72 | 42 |
| Coding | 94 | 1896 | ctggaacatctgatcctagc | 77 | 43 |
| Coding | 94 | 1083 | caaatgtctccacagtgatt | 79 | 44 |
| 3′UTR | 94 | 3270 | gctattgtgaacgaatgtca | 86 | 45 |
| 3′UTR | 94 | 3027 | caagattctagagtatgatg | 85 | 46 |
| Coding | 94 | 742 | atcatgtcatacctttcctt | 81 | 47 |
| Coding | 94 | 644 | gggaggctcttcctcttcat | 55 | 48 |
| Coding | 94 | 1782 | actgaagctttccacacaaa | 52 | 49 |
| Coding | 94 | 1016 | tcccacaaatgccattcctg | 78 | 50 |
| 3′UTR | 94 | 3563 | agaaaaccatttcttccttc | 75 | 51 |
| 3′UTR | 94 | 2973 | attattggcagctgattagt | 79 | 52 |
| Coding | 94 | 1933 | gcaccacattttgtgccttc | 74 | 53 |
| 3′UTR | 94 | 2962 | ctgattagtcagttttcaca | 85 | 54 |
| Coding | 94 | 2145 | tcaatgcagtattcatttca | 49 | 55 |
| Coding | 94 | 2167 | aagaagaccagaagtccgtc | 74 | 56 |
| Coding | 94 | 1365 | attcctttggagtaccacag | 81 | 57 |
| Coding | 94 | 1804 | ggtatctcttgtacattctc | 18 | 58 |
| Coding | 94 | 2419 | gggaactgctgaggttgctt | 46 | 59 |

In TABLE 1, the numbers in the % inhibition column are the percentage inhibition data reported in the '2466 publication for the antisense oligonucleotides described in Example 15 of the '2466 publication.

As indicated in TABLE 1, SEQ. ID NOs: 23-25, 27-30, 33-47, 50-54, 56 and 57 inhibited synthesis of human ADAM9 mRNA by at least 60% in quantitative real-time PCR experiments using the following PCR probes and primers:

(SEQ ID NO: 20)

| | |
|---|---|
| Forward Primer: | AGGGTTGGAAAATGATGGAAGA |

(SEQ ID NO: 21)

| | |
|---|---|
| Reverse Primer: | GATTCCGCAGGTTCCTCTCA |

(SEQ ID NO: 22)

| | |
|---|---|
| Probe: | FAM-CGGAGGTGGAGGCGACCGAGT-TAMRA |

as described in Examples 13-15 of US 2004/0092466, the disclosure of which is incorporated herein by reference.

In some embodiments, oligonucleotide ADAM9 inhibitory compounds have nucleotide sequences selected from SEQ ID NOS: 23-59.

In some embodiments, the oligonucleotide ADAM9 inhibitory compound will inhibit synthesis of ADAM9 mRNA by at least 70%, as measured in the quantitative real-time PCR assay described above. Specific examples of such ADAM9 inhibitory compounds include oligonucleotides having nucleotide sequences corresponding to SEQ ID NOS: 23, 24, 27, 30, 33, 36, 38, 39, 41-47, 50-54, 56, and 57.

In some embodiments, the oligonucleotide ADAM9 inhibitory compounds will inhibit synthesis of ADAM9 mRNA by at least 80%, as measured in the quantitative real-time PCR assay described above. Specific examples of such ADAM9 inhibitory compounds include oligonucleotides having sequences corresponding to SEQ ID NOS: 23, 27, 38, 39, 45, 46, 47, 50, 51, 54, and 57.

The sites or regions of the ADAM9-encoding nucleic acids to which oligonucleotide ADAM9 inhibitory compounds are complementary are referred to herein as “target sites” or “target regions.” The nucleotide sequences of such target sites or target regions are referred to herein as “target sequences.” The sequences of the target sites complementary to ADAM9 oligonucleotide inhibitory compounds having sequences corresponding to SEQ ID NOS: are provided in TABLE 2, infra, as SEQ ID NOS: 60-92.

TABLE 2

| | | | | |
|---|---|---|---|---|
| | | REV COMP | | SEQ |
| TARGET | | OF | | ID |
| SITE | SEQUENCE | SEQ ID | ACTIVE IN | NO |

| | | | | |
|---|---|---|---|---|
| 633 | aactgcaaaggatgaagagg | 23 | H. sapiens | 60 |
| 866 | agatttggaccaatggaaac | 24 | H. sapiens | 61 |
| 1119 | tgaattgggtcataatcttg | 25 | H. sapiens | 62 |
| 3357 | tcatggctataataaagcag | 27 | H. sapiens | 63 |
| 2769 | aataagtcttattcagtcat | 28 | H. sapiens | 64 |
| 2910 | ccttagttatcattaatgta | 29 | H. sapiens | 65 |
| 3375 | aggagcaattataaaatctt | 30 | H. sapiens | 66 |
| 1127 | gtcataatcttggaatgaat | 33 | H. sapiens | 67 |
| 3454 | ctttcaaagcttgctattaa | 34 | H. sapiens | 68 |
| 693 | agctgtcttgccacagaccc | 35 | H. sapiens | 69 |
| 1164 | ttgttcctgtggagcaaaga | 36 | H. sapiens | 70 |
| 444 | agttcataattcatccattg | 37 | H. sapiens | 71 |
| 2096 | gtgagactaaaggatacgga | 38 | H. sapiens | 72 |
| 3550 | taggctggagaaagaaggaa | 39 | H. sapiens | 73 |
| 2515 | cctttatatagttccctcac | 40 | H. sapiens | 74 |
| 2474 | ctcagggaaacttaattcct | 41 | H. sapiens | 75 |
| 3667 | ggtatggatcgtaaaatttt | 42 | H. sapiens | 76 |
| 1896 | gctaggatcagatgttccag | 43 | H. sapiens | 77 |
| 1083 | aatcactgtggagacatttg | 44 | H. sapiens | 78 |
| 3270 | tgacattcgttcacaatagc | 45 | H. sapiens | 79 |
| 3027 | catcatactctagaatcttg | 46 | H. sapiens | 80 |
| 742 | aaggaaaggtatgacatgat | 47 | H. sapiens | 81 |
| 644 | atgaagaggaagagcctccc | 48 | H. sapiens | 82 |
| 1782 | tttgtgtggaaagcttcagt | 49 | H. sapiens | 83 |
| 1016 | caggaatggcatttgtggga | 50 | H. sapiens | 84 |
| 3563 | gaaggaagaaatggttttct | 51 | H. sapiens | 85 |
| 2973 | actaatcagctgccaataat | 52 | H. sapiens | 86 |
| 1933 | gaaggcacaaaatgtggtgc | 53 | H. sapiens | 87 |
| 2962 | tgtgaaaactgactaatcag | 54 | H. sapiens | 88 |
| 2145 | tgaaatgaatactgcattga | 55 | H. sapiens | 89 |
| 2167 | gacggacttctggtcttctt | 56 | H. sapiens | 90 |
| 1365 | ctgtggtactccaaaggaat | 57 | H. sapiens | 91 |
| 2419 | aagcaacctcagcagttccc | 59 | H. sapiens | 92 |

By virtue of the demonstrated inhibitory activity of their respective complementary oligonucleotides, these targets have been experimentally found to be “open to,” and accessible for, hybridization with oligonucleotides. Those of skill will be able to design further embodiments of oligonucleotides capable of specifically hybridizing with these target sites to inhibit expression of ADAM9 using routine skill and experimentation. Accordingly, in some embodiments, the oligonucleotide ADAM9 inhibitory compound is an oligonucleotide capable of specifically hybridizing with a target sequence selected from SEQ ID NOS: 60-92 under physiological conditions.

Those of skill will also be able to identify additional target sites and sequences, and oligonucleotides capable of specifically hybridizing thereto, using routine skill and experimentation. Additional examples of antisense RNAs effective in reducing ADAM9 levels are described in Cisse et al., J. Biol. Chem. 280(49):40624-40631, incorporated herein by reference.

As used herein, the expression “oligonucleotide” is intended to encompass not only oligoribonucleic acids and oligo 2′-deoxyribonucleic acids, that are composed of naturally occurring and/or encoding nucleotide bases (“nucleobases,” e.g., adenine, guanine, thymine, cytosine and uracil) sugars and phosphate ester internucloside linkages, but also oligomers composed of one or more non-natural nucleobases, sugars and/or internucleoside linkages that retain their ability to specifically hybridize to a target site of an ADAM9-encoding nucleic acid. In some embodiments, such modified oligonucleotides are preferred over native forms owing to certain desirable properties, such as, for example, enhanced cellular uptake, enhanced affinity for the ADAM9-encoding nucleic acid under physiological or other conditions of use, and/or increased stability in the presence of degradatory enzyme, such as, for example, nucleases.

Representative examples of modified internucleoside linkages (“backbones”) that include a phosphorous atom and that can comprise the oligonucleotide ADAM9 inhibitory compounds include, but are not limited to, phosphorothioates, chiral phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, phosphinates, phosphoramidates including 3′-amino phosphoramidate, aminoalkylphosphoramidates, phosphorodiamidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′, or 2′ to 2′ linkage. In some embodiments, oligonucleotides having inverted polarity comprise a single 3′ to 3′ linkage at the 3′-most internucleotide linkage i.e., a single inverted nucleoside residue, which may be abasic (the nucleobase is missing or has a hydroxyl group in place thereof).

Methods of synthesizing oligonucleotides including one or more such modified internucleoside linkages are well-known. Specific representative methods are taught in U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated herein by reference.

Representative examples of modified backbones that do not include a phosphorous atom and that comprise the oligonucleotide ADAM9 inhibitory compounds include, but are not limited to, oligonucleotides in which the backbone is formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Methods of synthesizing oligonucleotides including one or more such modified internucleoside linkages are well-known. Specific representative examples are taught in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference.

Representative examples of modified nucleobases that comprise the ADAM9 oligonucleotide inhibitory compounds include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1 H-pyrimido[5,4-b][1,4]benzoxazin-2(3H)-one), phenothiazine cytidine (1H-pyrimido[5,4-b][1,4]benzothiazin; 2(3H)-one), G-clamps such as a substituted phenoxazine cytidine (e.g.

9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30:613, and those disclosed by Sanghvi Chapter 15, Antisense Research and Applications, pages 289-302, Crooke, S. T. and Lebleu, B. ed., CRC Press, 1993. Certain of these nucleobases are particularly useful for increasing the binding affinity of the oligonucleotide ADAM9 inhibitory compounds. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-12° C. (Sanghvi, et al., ed., Antisense Research and Applications, CRC Press, Boca Raton, 1993, pp. 276-278) and in some embodiments are preferred base substitutions, especially when combined with 2′-O-methoxyethyl sugar modifications.

Methods of synthesizing oligonucleotides including one or more modified nucleobases are well-known. Specific representative examples are taught in U.S. Pat. No. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, the disclosures of which are incorporated herein by reference.

Representative examples of modified sugars that can comprise the oligonucleotide ADAM9 inhibitory compounds include, but are not limited to, ribose that is substituted at the 2′-position with a group selected from OH, SH, SCH₃, OCN, CN, CF₃, OCF₃, SOCH₃, SO₂CH₃, ONO₂, NO₂, N₃,NH₂, Cl, Br, F, alkyl, O-alkyl, S-alkyl, NH-alkyl, alkenyl, O-alkenyl, S-alkenyl, NH-alkenyl, alkynyl, O-alkynyl, S-alkynyl, NH, alkynyl, O-alkyl-O-alkyl, alkyl, alkylaryl, O-alkylaryl, arylalkyl, O-arylalkyl, heterocycloalkyl, heterocycloalkylaryl, aminoalkylamino, polyalkylamino and substituted silyl where the alkyl group contains from 1-10 carbon atoms and the alkyl and alkynyl groups contain from 2-10 carbon atoms, and wherein the alkyl, alkenyl and alkynyl groups are optionally substituted. Specific examples of such 2′-substituent groups include, but are not limited to, O[(CH₂)ₙO]mCH₃, O(CH₂)ₙOCH₃,O(CH₂)ₙNH₂, O(CH₂)ₙCH₃, O(CH₂)ₙONH₂ and O[(CH₂)ₙON(CH₂)ₙCH₃]₂, where n and m are each, independently of one another, integers ranging from 1 to 10; OCH₂CH₂OCH₃, also known as 2′-methoxyethoxy, 2′-O-(2-methoxyethyl) or 2′-MOE (Martin et al., 1995, Helv. Chim. Acta. 78:486-504); OCH₂)₂ON(CH₃)₂, also known as 2′-DMAOE; OCH₂OCH₂N(CH₃)₂, also known as 2′-dimethyl-aminoethoxyethoxy, 2′-O-dimethyl 1-amino-ethoxy-ethyl or 2′-DMAEOE); OCH₃; OCH₂CH₂CH₂NH₂; allyl (CH₂CH═CH₂); O-allyl (O-CH₂CH═CH₂); and F.

The 2′-modification may be in the arabino (up) position or the ribo (down) position.

Further representative examples of modified sugars that can comprise the oligonucleotide ADAM9 inhibitory compounds include ribose substituted at the 3′-position or 5′-position of the terminal nucleotide with groups such as those exemplified above for the 2′-position, 2′-5′ linked ribose or deoxyribose sugars, cyclobutyl groups, etc.

Methods of synthesizing oligonucleotides including one or more such modified sugars are well-known. Specific representative examples are taught in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated herein by reference.

Another representative example of a modified sugar that can comprise the oligonucleotide ADAM9 inhibitory compound is ribose in which the 2′-hydroxyl group is linked to the 3′ or 4′-position, thereby forming a bicyclic sugar. The linkage bridging the 2′-oxygen atom and the 3′- or 4′-carbon atom is typically an alkylene group, for example, containing one or two methylene moieties (e.g., —CH₂— or —CH₂CH₂—). Oligonucleotides containing such bicyclic sugars are commonly referred to as locked nucleic acids or “LNAs.” Methods for synthesizing LNAs are described in WO 98/39352 and WO 99/14226, the disclosures of which are incorporated herein by reference.

In some embodiments, modified oligonucleotides include compounds in which both the sugar and phosphate ester groups are replaced with non-sugar phosphate linkages (referred to herein as “oligonucleotide mimetics”). The nucleobases are retained for hybridization with the ADAM9-encoding nucleic acid. One representative example of such an oligonucleotide mimetic that has been shown to have excellent hybridization and other properties is referred to as a peptide nucleic acid (“PNA”). In PNAs, the sugar-phosphate backbone of an oligonucleotide is replaced with an amide containing backbone, the most common of which is an aminoethylglycine backbone (see, e.g., Nielsen et al., 1991, Science 254:1497-1500). Methods of synthesizing PNA compounds are described, for example, in U.S. Pat. Nos. 5,539,082; 5,714,331; and 5,719,262, the disclosures of which are incorporated herein by reference.

In some embodiments, oligonucleotide ADAM9 inhibitory compounds are oligonucleotides with phosphorothioate internucleosidic linkages, heteroatomic internucleosidiclinkages or combinations thereof. Specific exemplary heteroatomic interlinkages include, but are not limited to, —CH₂—NH—O—CH₂—, —CH₂—N(CH₃)—O—CH₃— (known as a methylenene (methylimino) or MMI backbone, —CH₂—O—N(CH₃)—CH₂—CH₂, amides and morpholino. These internucleoside backbones, as well as methods of synthesizing oligonucleotides including them, are described, for example, in U.S. Pat. Nos. 5,489,677; 5,602,240; and 5,034,506, the disclosures of which are incorporated herein by reference.

Oligonucleotide ADAM9 inhibitory compounds can include one or more moieties or conjugates covalently bound to appropriate functional groups of the oligonucleotide moiety of the compound. Conjugate groups or moieties can be selected to impart the oligonucleotide ADAM9 inhibitory compound with specified properties as compared to an un-conjugated oligonucleotide, such as, for example, cellular uptake, cellular distribution, cellular activity, pharmacokinetic activities, etc. Exemplary conjugating groups include, but are not limited to, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the disclosure of which is incorporated herein by reference. Conjugate moieties include but are not limited to lipid moieties such as a cholesterol moiety (Letsinger et al., 1989, Proc. Natl. Acad. Sci. USA 86:6553-6556), cholic acid (Manoharan et al., 1994, Bioorg. Med. Chem. Let. 4:1053-1060), a thioether, e.g., hexyl-S-tritylthiol (Manoharan et al., 1992, Ann. N.Y. Acad. Sci., 660:306-309; Manoharan et al., 1993, Bioorg. Med. Chem. Let. 3:2765-2770), a thiocholesterol (Oberhauser et al., 1992, Nucl. Acids Res. 20:533-538), an aliphatic chain, e.g., dodecandiol or undecyl residues (Saison-Behmoaras et al., 1991, EMBO J. 10:1111-1118; Kabanov et al., 1990, FEBS Lett., 259 327-330; Svinarchuk et al., 1993, Biochimie 75:49-54), a phospholipid, e.g., di-hexadecyl-rac-glycerol or triethyl-ammonium 1,2-di-O-hexadecyl-rac-gly-cero-3-H-phosphonate (Manoharan et al., 1995, Tetrahedron Lett., 36:3651-3654; Shea et al., 1990, Nucl. Acids Res. 18:3777-3783), a polyamine or a polyethylene glycol chain (Manoharan et al., 1995, Nucleosides & Nucleotides 14:969-973), or adamantane acetic acid (Manoharan et al., 1995, Tetrahedron Lett. 36:3651-3654), a palmityl moiety (Mishra et al., 1995, Biochim. Biophys. Acta 1264:229-237), or an octadecylamine or hexylamino-carbonyl-oxycholesterol moiety (Crooke et al., 1996, J. Pharmacol. Exp. Ther. 277, 923-937). Oligonucleotide ADAM9 inhibitory compounds may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. Pat. No. 6,656,730 (filed Jun. 15, 1999) which is incorporated herein by reference in its entirety.

Representative patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, the disclosures of which are incorporated herein by reference.

It is not necessary for all positions in a given oligonucleotide ADAM9 inhibitory compound to be uniformly modified, and in fact more than one of the aforementioned modifications may be incorporated in a single compound or even at a single nucleoside within an oligonucleotide. The oligonucleotide ADAM9 inhibitory compounds also include oligonucleotides which are chimeric compounds. “Chimeric” oligonucleotides or “chimeras,” in the context of this disclosure, are oligonucleotides that contain two or more chemically distinct regions, each made up of at least one monomer unit, i.e., a nucleotide in the case of an unmodified oligonucleotide compound. These oligonucleotides typically contain at least one region wherein the oligonucleotide is modified so as to confer upon the oligonucleotide increased resistance to nuclease degradation, increased cellular uptake, increased stability and/or increased binding affinity for the target nucleic acid. An additional region of the oligonucleotide may serve as a substrate for enzymes capable of cleaving RNA:DNA or RNA:RNA hybrids. By way of example, RNAse H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. The cleavage of RNA:RNA hybrids can, in like fashion, be accomplished through the actions of endoribonucleases, such as interferon-induced RNAseL which cleaves both cellular and viral RNA. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligonucleotides can be synthesized as composite structures of two or more oligonucleotides, modified oligonucleotides, oligonucleosides and/or oligonucleotide mimetics as described above. Such compounds have also been referred to in the art as hybrids or gapmers. Representative U.S. patents that teach the synthesis of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, the disclosures of which are incorporated herein by reference.

The oligonucleotide ADAM9 inhibitory compounds described herein may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

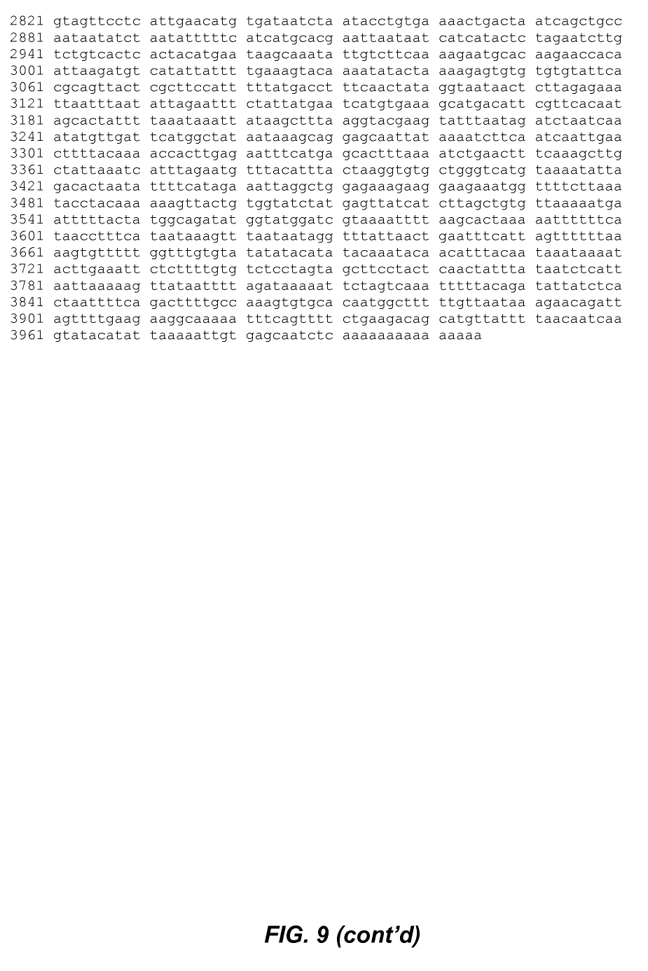

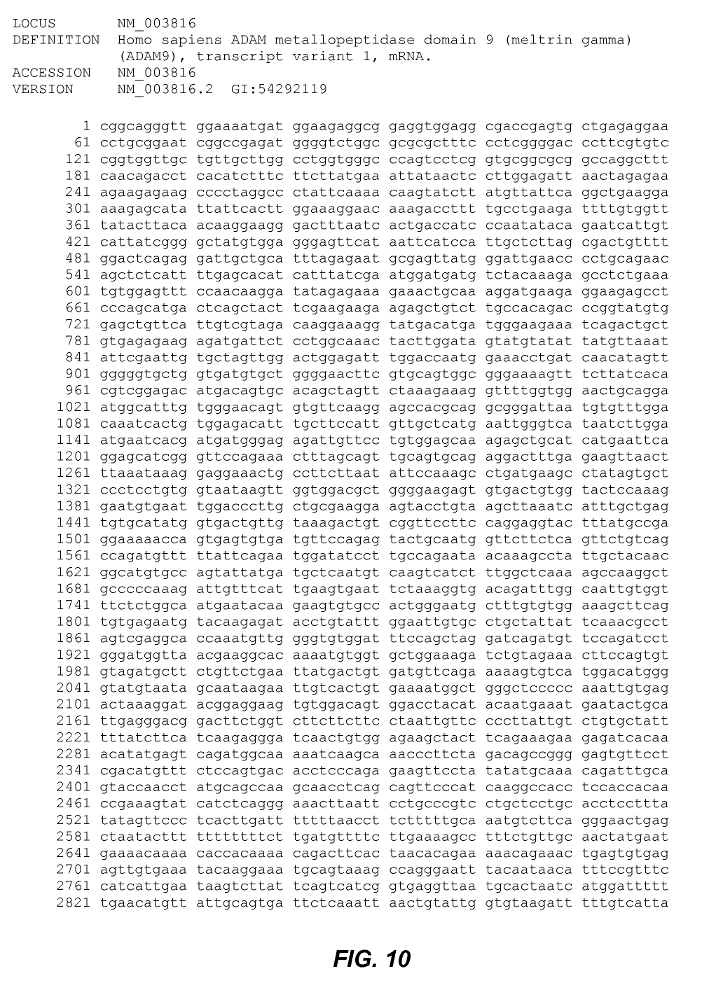

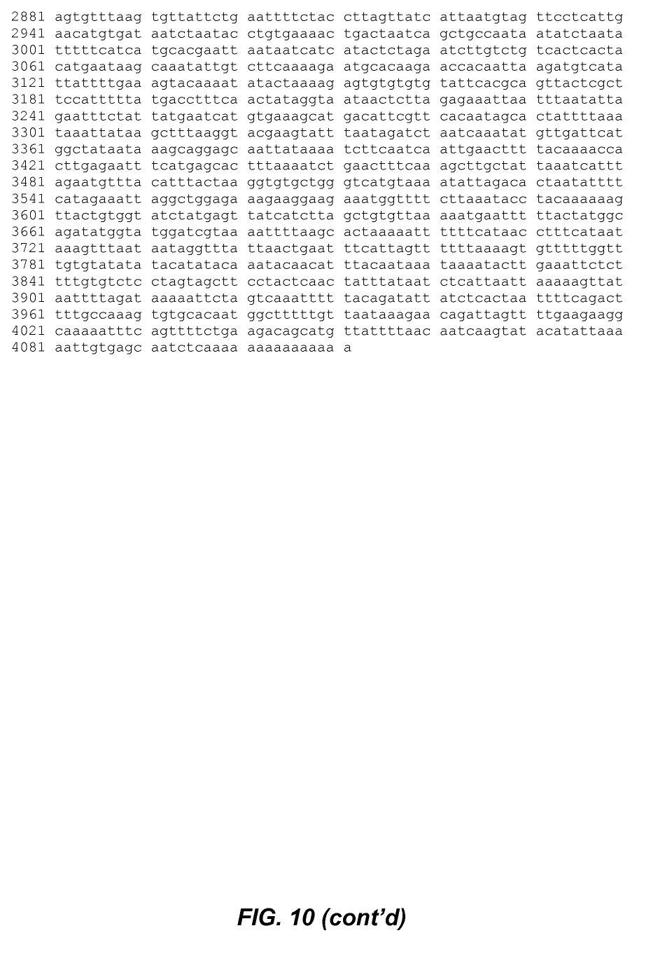

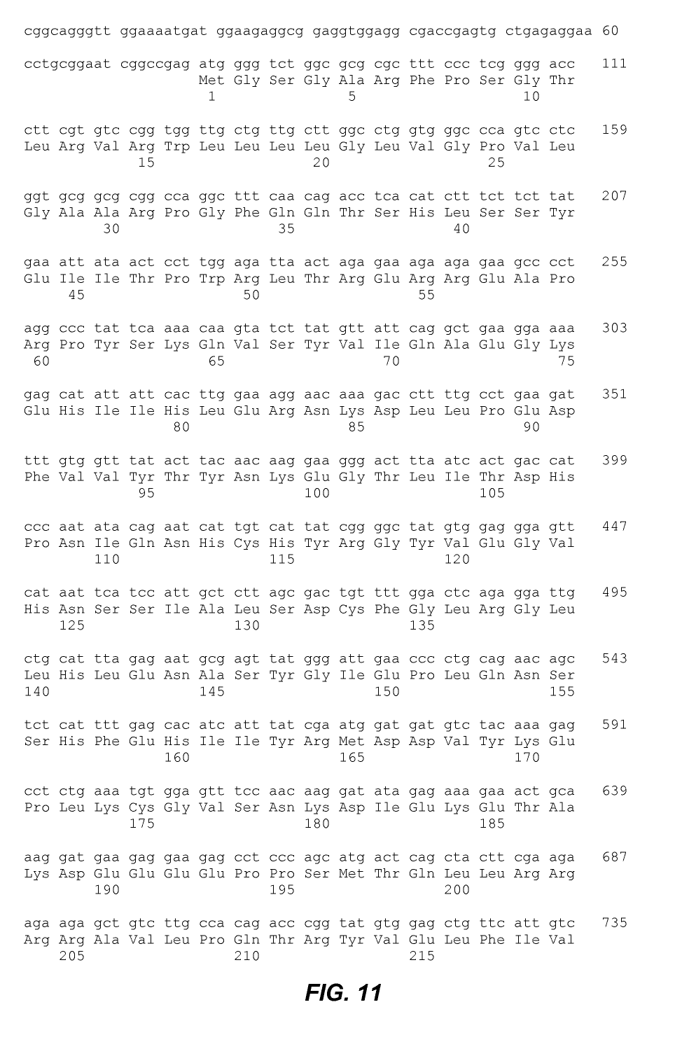

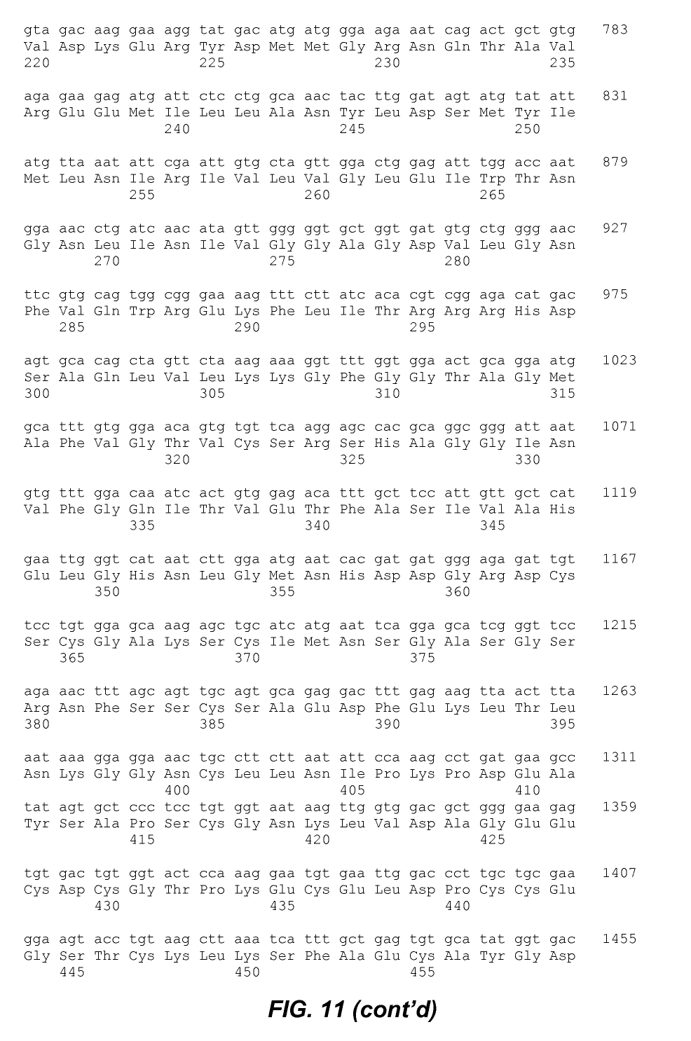

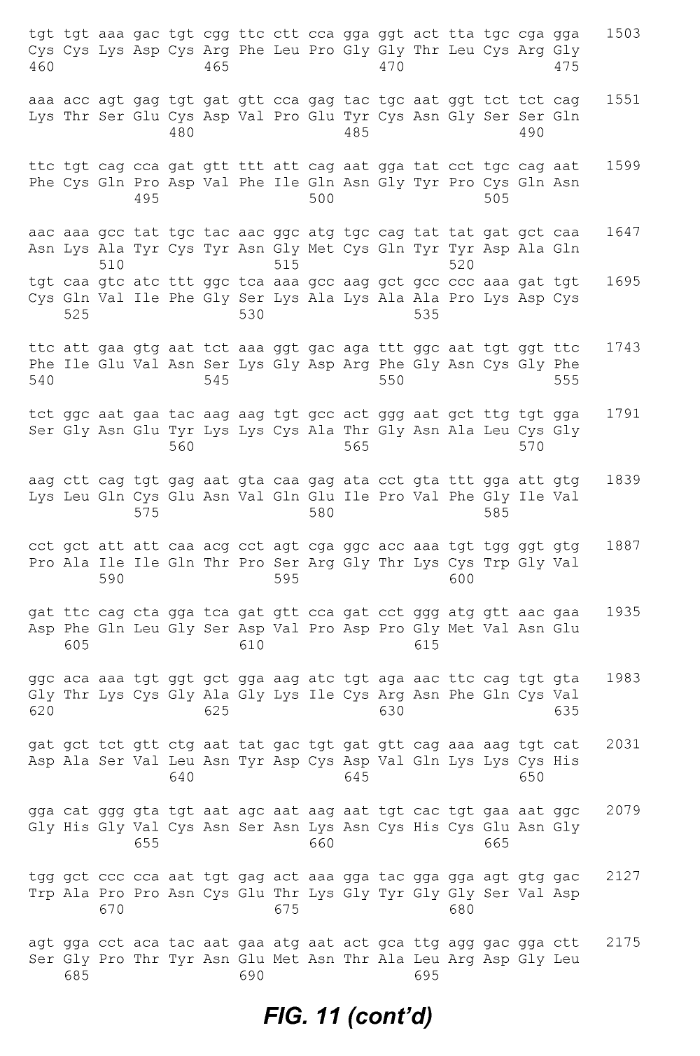

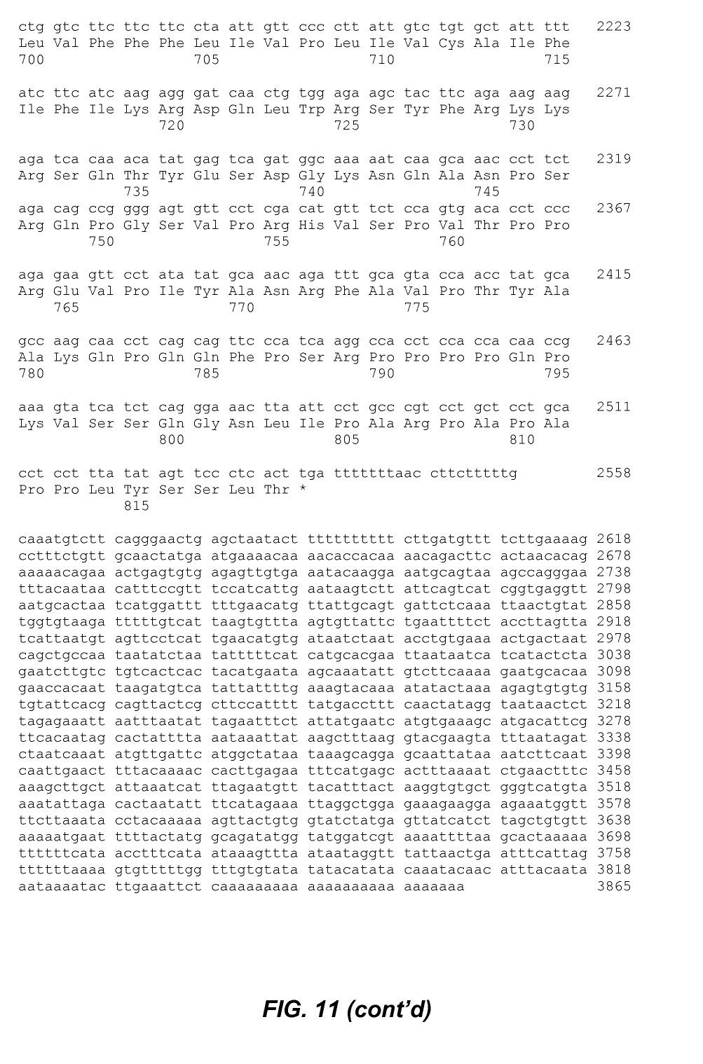

As noted above, methods of determining amounts of mRNA gene expression products are well-known, and include by way of example and not limitation, microarray analyses (Brazma and Vilo, 2000, FEBS Lett. 480:17-24; Celis et al., 2000, FEBS Lett. 480:2-16), serial analysis of gene expression (“SAGE;” Madden et al., 2000, Drug Discovery Today 5:415-425), restriction enzyme amplification of digested cDNAs (“READS;” Prashar & Weissman, 1999, Methods Enzymol. 303:258-272), total gene expression analysis (“TOGA;” Sutcliffe et al., 2000, Proc. Nat'l Acad. Sci. USA 97:1976-81), expressed sequence tag sequencing (Celis et al., 2000, FEBS Lett. 480:2-16; Larsson et al., 2000, J. Biotechnol. 80:143-157), subtractive RNA fingerprinting (“SURF;” Fuchs et al., 2000, Anal. Biochem. 286:91-98; Larson et al., 2000, Cytometry 41:203-208), subtractive cloning differential display (“DD;” Jurecic and Belmont, 2000, Curr. Opin. Microbiol. 3:316-321), comparative genomic hybridization (Carulli et al., 1998, J. Cell. Biochem. Supp. 31:286-96) and quantitative real-time RT PCR (“A-Z of Quantitative PCR,” Bustin, Ed., INL Biotechnology Series, International University Line, La Jolla, Calif. 2004). Reagents for carrying out these assays can be designed from the nucleotide sequences illustrated in FIGS. 9, 10 and 94 using well-known, routine means.

Non-oligonucleotide ADAM9 inhibitory compounds described herein can likewise be synthesized using standard techniques. For example, small organic ADAM9 inhibitory compounds can be synthesized using standard techniques of organic chemistry. Protecting groups suitable for use in such syntheses are described, for example, in Greene and Wuts, “Protective Groups in Organic Chemistry,” John Wiley and Sons, Inc., 1999.

The ADAM9 inhibitory compounds will generally exhibit IC₅₀s of ADAM9 protease activity of less than about 1 mM, as measured in a standard in vitro inhibition assay, such as, for example the ADAM9 inhibition assay described herein, although ADAM9 inhibitory compounds that exhibit higher IC₅₀s may also find use in the methods and compositions described herein. In some embodiments, such ADAM9 inhibitory compounds will exhibit IC₅₀s in the range of 100 μM, 75 μM, 50 μM, 25 μM, 10 μM, 1 μM, 100 nM, 75 nM, 50 nM, 25 nM, 10 nM, 1 nM, or even lower. As will be appreciated by skilled artisans, in many contexts, ADAM9 inhibitory compounds having IC₅₀s in the micromolar, nanomolar or even subnanomolar range may be desirable.

The suitability of any compound that inhibits the metalloprotease activity of a matrix metalloprotease, including the above-described compounds for use as an ADAM9 inhibitory compound in the compositions and methods as described herein can be accessed in assays with ADAM9.

In some embodiments, the assay for inhibitors of ADAM9 can be based on its effect on retinal neovascularization OIR model, comprising administering to an eye of an animal a compound capable of inhibiting ADAM9, subjecting the animal to OIR, and measuring pathological neovascularization in the eye. Characteristics such as enlarged central asvascular areas and development of retinal vasculature can be examined. In some embodiments, the angiogenesis can be assayed based on a matrix gel that when introduced into a host animal provides a scaffold for blood vessel formation (see, e.g., U.S. Pat. No. 5,382,514). The ADAM9 inhibitory agent can be introduced into the matrix gel and the level of angiogenesis in the gel quantitated. In some embodiments, the matrix gel can be introduced along with cells that overexpress ADAM9 to promote angiogenesis in the matrix.

In some embodiments, the assay for inhibitors of ADAM9 can use polypeptide targets that are recognized and cleaved by ADAM9, or whose proteolysis is indirectly increased by ADAM9 activity. For substrates that are recognized and cleaved by ADAM9, an ADAM9 polypeptide can be contacted with an ADAM9 target polypeptide in presence of an inhibitor, and the presence of the proteolytic product measured. In some embodiments, where proteolysis of the polypepeptide target is directly or indirectly affected by ADAM9, a cell based system can be used where the polypeptide target is expressed along with ADAM9 and the cells are incubated with an inhibitor and the proteolytic product detected. In some embodiments, the polypeptide targets can be selected from one or more of FGFR2iiib, EGF (Peduto et al., 2005, Cancer Research 65:9312-9319), CD40, EphB4, Flk-1, Tie-2, VE-cadherin and VCAM. As further described in detail below, use of ADAM9 target polypeptides can provide a screening method for identifying inhibitors of ADAM9 protease activity, and therefore inhibitors of ADAM9 mediated angiogenesis.

The ADAM9 inhibitory compounds may be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative U.S. patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, the disclosures of which are incorporated herein by reference.

The ADAM9 inhibitory compounds described herein encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the ADAM9 inhibitory compounds, compounds pharmaceutically acceptable salts of such prodrugs, and other bioequivalents.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, e.g., for example, Berge et al., 1977, “Pharmaceutical Salts,” J. of Pharma Sci. 66:1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions described herein. These include organic or inorganic acid salts of the amines. In some embodiments, acid salts are hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotide ADAM9 inhibitory compounds, specific examples of pharmaceutically acceptable salts include, but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

When administered to patients as a therapeutic approach towards the treatment of pathological neovascularization, the ADAM9 inhibitory compounds can be administered as the compound per se, or in the form of pharmaceutical compositions.

The compounds and/or pharmaceutical compositions may be administered in a number of ways depending upon whether local or systemic treatment is desired.

Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; intracranial, e.g., intrathecal or intraventricular; intraocular, and intravitreal administration. Oligonucleotide ADAM9 inhibitory compounds with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Preferred topical formulations include those in which the oligonucleotides of the invention are in admixture with a topical delivery agent such as lipids, liposomes, fatty acids, fatty acid esters, steroids, chelating agents (e.g., β-cyclodextrins) and surfactants. Preferred lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). ADAM9 inhibitory compounds may be encapsulated within liposomes or may form complexes thereto, in particular to cationic liposomes. Alternatively, the compounds may be complexed to lipids, for example cationic lipids. Exemplary fatty acids and esters include but are not limited to, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which the inhibitor is administered in conjunction with one or more penetration enhancers surfactants and chelators. Exemplary surfactants include fatty acids and/or esters or salts thereof, bile acids and/or salts thereof. Exemplary bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusid-ate and sodium glycodihydrofusidate. Exemplary fatty acids include undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). In some embodiments, oligonucleotide or other ADAM9 inhibitory compounds are administered with combinations of penetration enhancers, for example, fatty acids/salts in combination with bile acids/salts. An exemplary combination is the sodium salt of lauric acid, capric acid and UDCA. Further penetration enhancers include polyoxyethylene-9-lauryl ether, polyoxyethylene-20-cetyl ether.

ADAM9 inhibitory compounds may be delivered orally, in granular form including sprayed dried particles, or complexed to form micro or nanoparticles. For oligonucleotide ADAM9 inhibitory compounds, complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

As will be appreciated by skilled artisans, ADAM9 inhibitory compounds that are biological in nature, for example, peptides, polypeptides, oligonucleotides and polynucleotides, can be administered as the inhibiting compound per se, or alternatively nucleic acids encoding such inhibitory compounds can be administered, and the inhibitory compound expressed, either by transcription or translation, in vivo. Methods for administering nucleic acids encoding entire therapeutic agents are well known, and are described, for example, in US Patent Application Nos. 20040086486 and 20020151516, the disclosures of which are incorporated herein by reference. Uptake of nucleic acids by mammalian cells may be enhanced using several known transfection techniques including the use of transfection agents. The formulation which is administered may contain such agents. Example of these agents include cationic agents (for example calcium phosphate and DEAE-dextran) and lipofectants (for example LIPOFECTAM™ and TRANSFECTAM™).

The amount of ADAM9 inhibitory compound administered will depend upon a variety of factors, such as the mode of administration, the activity of the compound, the height, age, weight and general condition etc., of the patient, the bioavailability of the compound, the type and/or location of the condition to be treated, and other criteria apparent to the treating physician. Typically, an amount of compound effective to achieve a desired level of ADAM9 inhibition in the target tissue will be administered. Initial dosages can be estimated initially from in vitro assays and adjusted for specific desired ADAM9 inhibitory compounds using routine methods. For example, an initial dosage for use in animals may be formulated to achieve a circulating blood or serum concentration of active compound that inhibits about 25% or more of ADAM9 activity, or a process associated therewith, such as processing of EphB4 by overexpressed ADAM9, as measured in an in vitro cell based assay. Alternatively, an initial dosage for use in animals may be formulated to achieve a circulating blood or serum concentration of active compound that is equal to or greater than the IC₅₀ as measured in an in vitro assay. A suitable dose may be from 0.1 to 100 mg/kg body weight such as 1 to 40 mg/kg body weight. Calculating dosages to achieve such circulating blood or serum concentrations taking into account the bioavailability of the particular active compound is well within the capabilities of skilled artisans. For guidance, the reader is referred to Fingl and Woodbury, “General Principles,” In: The Pharmaceutical Basis of Therapeutics, Chapter 1, pp. 1-46, 1975, and the references cited therein.

In addition to the foregoing, the present disclosure provides a method of screening for ADAM9 activity, particularly methods of screening and identifying inhibitors of ADAM9 activity that can be used in reducing or inhibiting pathological neovascularization and/or angiogenesis. As noted above, since ADAM9 is a metalloprotease, which has been implicated in the processing of membrane proteins such as EGF or the FGFR2iiib (Horiuchi et al., 2007. Mol. Biol. Cell. 18:176-188; Peduto et al., 2005, Cancer Res 65:9312-9319), polypeptides that are associated with angiogenesis, particularly membrane proteins, were examined as targets for ADAM9 activity. ADAM9 along with candidate target polypeptides were co-expressed in a cell and the release of candidate substrate proteins measured as compared to a cell overexpressing the catalytically inactive ADAM9 E>A mutant as a control. It was found that shedding or release of CD40, EphB4, Flk-1, Tie-2, VE-cadherin and VCAM from the cells was increased by overexpression of ADAM9 compared to the inactive ADAM9 E>A mutant. Although ADAM9 presumably has more substrates on the surface of endothelial cells than the proteins examined herein, and other non-catalytic protein modules, such as the disintegrin-domain and cysteine-rich region of ADAM9, or its cytoplasmic domain, could also be important for the function of ADAM9 in neovascularization, the observations herein provide a basis for screening and identifying candidate compounds as inhibitors of ADAM9 activity, and thus inhibitors of ADAM9 mediated neovascularization and angiogenesis.

In some embodiments, a method of screening for an agent that inhibits ADAM9 mediated angiogenesis can comprise contacting ADAM9 polypeptide with an ADAM9 target polypeptide in presence of a candidate agent, and determining the presence of proteolyzed target polypeptide. As used herein, an “ADAM9 target polypeptide” refers to a polypeptide that is subject to proteolysis or cleavage by ADAM9 protease. In some embodiments, target polypeptide comprises at least one or more of polypeptides FGFR2iiib, EGF, CD40, EphB4, Flk1, Tie-2, VE-cadherin and VCAM.

“FGFR2iiib”, also known as fibroblast growth factor receptor 2 IIIb or FGFR2, refers to a member of the fibroblast growth factor receptor family. A representative full-length protein consists of an extracellular region composed of three immunoglobulin-like domains, a single hydrophobic membrane-spanning segment, and a cytoplasmic tyrosine kinase domain. FGFR2iiib has high-affinity for acidic, basic and/or keratinocyte growth factor. The amino acid and nucleic acid sequences for human FGFR2iiib are disclosed as Swiss-Pro/Genbank Accession Nos. M97193.1, NM_{—}000141.4, and NM_{—}022970.3, and in Dell et al., 1992, J. Biol. Chem. 267(29):21225-21229. The amino acid and nucleic acid sequences for FGFR2 from other organisms are also known and may be used in the assays, e.g., dog, (NM_{—}001003336.1 and NP_{—}001003336.1); chimpanzee (XM_{—}001157227.1 and XP_{—}001157227.1), rat (XM_{—}341940.3 XP_{—}341941.3) and mouse (NM_{—}010207.2, and NP_{—}034337.2).

“EGF” refers to epidermal growth factor (beta-urogastrone), which stimulates the growth of various epidermal and epithelial tissues in vivo and in vitro and of some fibroblasts in cell culture through interaction of it cognate receptor, EGFR/ErbB 1. The amino acid and nucleic acid sequences for human EGF are disclosed as Swiss-Pro/Genbank Accession Nos. P01133, NM_{—}001963.3, and NP_{—}001954.2. The amino acid and nucleic acid sequences for EGF from other organisms are also known and may be used in the assays, e.g., dog (NM_{—}001003094.1 and NP_{—}001003094.1), chimpanzee (XM_{—}517395.2 and XP_{—}517395.2), cow (XM_{—}001253862.1 and XP_{—}001253863.1), rat (NM_{—}012842.1 and NP_{—}036974.1), and mouse (NM_{—}010113.21 and NP_{—}034243.11).

“CD40” refers to a member of the TNF-receptor superfamily (TNF receptor superfamily member 5) and is a costimulatory protein found on antigen presenting cells. It is essential in mediating a broad variety of immune and inflammatory responses including T cell-dependent immunoglobulin class switching, memory B cell development, and germinal center formation. Adaptor protein TNFR2 interacts with this receptor and serves as a mediator of the signal transduction. The amino acid and nucleic acid sequences for human CD40 are disclosed as Swiss-Pro/Genbank Accession Nos. P25942, NM_{—}001250.4, and NM_{—}152854.2. The amino acid and nucleic acid sequences for CD40 from other organisms are also known and may be used in the assays, e.g., dog (NM_{—}001002982.1 and NP_{—}001002982), cow (XM_{—}581509.3 and XP_{—}581509.2), rat (NM_{—}134360.1 and NP_{—}599187.1), and mouse (NM_{—}011611.21 and NP_{—}035741.21).

“EphB4” , also known as HTK, refers to EPH receptor, which is a transmembrane protein. EphB4 binds to ephrin-B2 and plays an essential role in vascular development (Flanagan et al., 1998, Annu. Rev. Neurosci. 21:309-45). It has tyrosine kinase activity and is expressed in primary CD34+ hematopoietic progenitors and other myeloid cells. The amino acid and nucleic acid sequence for EphB4 are disclosed as Swiss-Pro/Genbank Accession Nos. P54760 and NM_{—}004444.4, and also disclosed in Bennett et al., 1994, J Biol Chem. 269:14211-14218. The amino acid and nucleic acid sequences for EphB4 from other organisms are also known and may be used in the assays, e.g., dog (XM_{—}546948.2 and XP_{—}546948.2), chimpanzee (XM_{—}519269.2 and XP_{—}519269.2), cow (XM_{—}869400.2 and XP_{—}874493.2) and mouse (NM_{—}010144.4, and NP_{—}034274.3).

“Flk1” , also known as kinase insert domain receptor (KDR) and vascular endothelial growth factor receptor 2 (VEGFR2), is a member of the protein tyrosine kinase superfamily and plays a key role in vascular development and regulation of vascular permeability. The polypeptide contains a protein kinase domain and seven Ig-like C2-type (immunoglobulin-like) domains. The amino acid and nucleic acid sequences for the human Flk1 are disclosed in Swiss-Pro/Genbank Accession Nos. P35968 and NM_{—}002253.2. The amino acid and nucleic acid sequences for EphB4 from other organisms are also known and may be used in the assays, e.g., dog (XM_{—}539273.2 and XP_{—}539273.2), chimpanzee (XM_{—}517284.2 and XP_{—}517284.2), cow (XM_{—}611785.3 and XP_{—}611785.3) and mouse (NM_{—}010612.2 and NP_{—}034742.2).

“Tie-2”, also referred to as angiopoietin-1 receptor, is a receptor tyrosine kinase that is expressed mainly in endothelial cells in mice, rats, and humans. The receptor has an extracellular domain containing 2 immunoglobulin-like loops separated by 3 epidermal growth factor-like repeats that are connected to 3 fibronectin type III-like repeats. The ligand for the receptor is angiopoietin-1. The amino acid and nucleic acid sequence for the human Tie-2 are disclosed in Swiss-Pro/Genbank Accession Nos. Q02763 and NM_{—}000459.3. The amino acid and nucleic acid sequences for Tie-2 from other organisms are also known and may be used in the assays, e.g., chimpanzee (XM_{—}520519.2 and XP_{—}520519.2), cow (NM_{—}173964.2 and NP_{—}776389.1), rat (XM_{—}342863.3 and XP_{—}342864.3), and mouse (NM_{—}013690.21 and NP_{—}038718.21).

“VE-cadherin”, also known as CDH5, is a member of the cadherin superfamily. It is a calcium-dependent cell-cell adhesion glycoprotein comprised of five extracellular cadherin repeats, a transmembrane region and a highly conserved cytoplasmic tail. Like other cadherins, it imparts to cells the ability to adhere in a homophilic manner and may play an important role in endothelial cell biology through control of the cohesion and organization of the intercellular junctions. The amino acid and nucleic acid sequences for the human VE-cadherin are disclosed in Swiss-Pro/Genbank Accession Nos. P33151 and NM_{—}001795.3. The amino acid and nucleic acid sequences for VE-cadherin from other organisms are also known and may be used in the assays, e.g., dog (XM_{—}546894.2 and XP_{—}546894.2), chimpanzee (XM_{—}523383.2 and XP_{—}523383.2), cow (NM_{—}001001601.1 and NP_{—}001001601.1), rat (XM_{—}226213.4 and XP_{—}226213.4), and mouse (NM_{—}009868.31 and NP_{—}033998.21).

“VCAM”, also known as vascular cell adhesion protein 1 or CD106 refers to a member of the Ig superfamily characterized as a cell surface sialoglycoprotein expressed by cytokine-activated endothelium. This type I membrane protein mediates leukocyte-endothelial cell adhesion and signal transduction, and may play a role in the development of artherosclerosis and rheumatoid arthritis. The amino acid and nucleic acid sequence for the human VE-cadherin are disclosed in Swiss-Pro/Genbank Accession Nos. Nos. P19320, NP_{—}001069.1, and NP_{—}542413.1. The amino acid and nucleic acid sequences for VCAM from other organisms are also known and may be used in the assays, e.g., dog (NM_{—}001003298.1 and NP_{—}001003298.1), cow (XM_{—}614390.3 and XP_{—}614390.2), rat (NM_{—}012889.1 and NP_{—}037021.1 ), and mouse (NM_{—}011693.21 and NP_{—}035823.21).

In some embodiments, the target polypeptide comprises at least one or more of CD40, EphB4, Flk1, Tie-2, VE-cadherin and VCAM-1, which have not been previously identified as being targets for ADAM9 activity. In some embodiments, the method includes at least the target polypeptide EphB4. In some embodiments, in addition to one or more of foregoing, the suite of target polypeptides includes one or more of FGFR2iiib and EGF.

In some embodiments, the target polypeptides can be contacted with a preparation of ADAM9 protease in-vitro that contains ADAM9 and target polypeptide, or in a cell-free system using cell extracts that contain the target polypeptides, either present endogenously or added exogenously from preparation of the target polypeptides. These in-vitro or cell-free based assays are particularly useful where the target polypeptides are direct substrates for ADAM9 protease activity.

In some embodiments, the ADAM9 polypeptide and the target polypeptides can be co-expressed in a cell, and the cell incubated with the candidate agent. Typically, the cell can be incubated with the candidate agent prior to the assay for release of the proteolyzed target peptide by ADAM9 over a time course of 30 minutes to 6 hours. Where the assays are based on co-expression of ADAM9 and target polypeptides in a cell, polynucleotide constructs that are capable of expressing the polypeptides can be introduced into a cell by various techniques known in the art to allow transient expression of the polypeptides. In some embodiments, cells that have been polynucleotide constructs that have inserted into the host cell chromosome can be used.

The polynucleotide constructs for expressing ADAM9 and/or ADAM9 target polypeptides can use a regulated expression system for controlling the expression of ADAM9 and/or the target polypeptides. Regulatable systems can be based on those typically used in the art, such as, among others, cumate inducible systems (see, e.g., Mullick et al., 2006, BMC Biotechnol. 6:43); tet regulated systems Gossen et al., 1992, Proc. Natl. Acad. Sci. USA 89:5547-5551; lac opertors based systems (see, e.g., Fire et al., 1990, Gene 93(2):189-98); and ecdyson regulated systems (e.g., Moradpour et a., 1998, Biol Chem. 379(8-9):1189-91). Other inducible systems will be apparent to the skilled artisan. Guidance on eukaryotic expression vectors is provided in Ausubel. F., Current Protocols in Molecular Biology, Green Associates Pub, 1998 (updates to 2008); Sambrook et al., 2001, Molecular Cloning: A Laboratory Manual, 3rd Ed., Cold Spring Harbor Laboratory Press; and Methods in Enzymology, Vol. 306, Glorioso et al. eds., Academic Press, 1^{st} Ed., 1999. All references are incorporated herein by reference.

Since many of the target polypeptides are membrane bound, the proteolyzed products are released from the cell and the released products can be detected by a suitable method. The proteolyzed products can be detected by any number of techniques, such as by antibodies that specifically bind to target polypeptides or by having a detectable moiety (e.g., a detectable label) that is bound to the target polypeptide. For embodiments using a detectable moiety, the label may be a direct label, i.e., a label that itself is detectable or produces a detectable signal, or it may be an indirect label, i.e., a label that is detectable or produces a detectable signal in the presence of another compound. The method of detection will depend upon the labeled used, and will be apparent to those of skill in the art.

In some embodiments, the detectable moiety is a direct label, including, among others, radiolabels, fluorophores, chromophores, chelating agents, particles, chemiluminescent agents. Suitable radiolabels include, by way of example and not limitation, ³H, ¹⁴C, ³²P, ³⁵S, ¹²⁵I, ¹³¹I and ¹⁸⁶Re. Suitable fluorophores include, by way of example and not limitation, fluorescein, rhodamine, phycoerythrin, Texas red, free or chelated lanthanide series salts such as Eu³⁺ and the myriad fluorophores available from Molecular Probes Inc., Eugene, Oreg. Examples of suitable colored labels include, by way of example and not limitation, metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as those described Snyder (EP 0 280 559 and 0 281 327) and dyes encapsulated in liposomes as described by Campbell et al. (U.S. Pat. No. 4,703,017).

In some embodiments, the detectable moiety is an antibody tag that is detectable with any antibody specific for the tag. Any suitable peptide sequences for which antibodies are available or to which antibodies can be made are useful as epitope tags. Exemplary epitope tags, include, among others, FLAG tag, c-myc tag (C-MYC), hemagglutinin (HA), GST tag, and 6XHIS tags (see, e.g., Kroll et al., 1993, DNA Cell Biol. 12:441-453; di Guan et al., 1988, Gene 67:21-30; Guan et al., 1991, Anal Biochem. 192:262-267; Davis et al., 1993, Biotechnology 11:933-936; Yu et al., 1998, Mol. Cell. Biol. 18:1379-1387; Field et al., 1988, Mol. Cell. Biol. 8:2159-2165; Munro et al., 1984, EMBO J. 3:3087-3093; and Brizzard et al., 1994, Biotechniques 16:730-735).

In some embodiments, the detectable moiety is a detectable reporter protein, such as an enzyme or fluorescent protein. In some embodiments, a detectable enzyme can be attached chemically to the target polypeptides while in some embodiments, the detectable enzyme can be expressed with the target polypeptide as a fusion protein. Preferably, where the detectable enzyme is expressed as a fusion protein in a cell-based assay, the fusion is constructed such that the proteolyzed target polypeptide portion released from the cell retains the detectable enzyme. A variety of suitable enzyme labels can be used, including, among others, acid phosphatase, alkaline phospatase (e.g., bacterial, placental, shrimp), luciferase, β-galactosidse (β-GAL), β-glucouronidase; and chloramphenicol acetyltransferase (CAT). Description and guidance on various reporter genes as well as methods for detecting enzyme activity are provided in Reporter Gene: A Practical Guide, Humana Press, Anson et., 2007. Other enzyme based labels that may be used will be apparent to those of skill in the art.

In some embodiments, the reporter protein is a fluorescent protein that can be expressed as a fusion protein with the target polypeptides. As noted above, the fusion can be constructed such that the proteolyzed target polypeptide portion released from the cell retains the fluorescent protein. Various fluorescent proteins, such as green fluorescent protein, Clavula fluorescent protein, including mutants with different absorption and emission characteristics can be used to detect the proteolytic activity of ADAM9. Such fluorescent proteins as well as their amino acid sequences, and spectral characteristics can be found in, among others, U.S. Pat. Nos. 5491084, 5625048, 5777079, 5804387, 6064321, 6096865, 6194548, 6414119, 6780975, 6969597, and 7015310. All publications are incorporated herein by reference.

In some embodiments, the detectable moiety bound to each type of target polypeptide are distinguishable. For example, different enzyme-based reporter gene can be bound to each of the different ADAM9 target polypeptides such that the proteolytic products of each of the target polypeptides can be distinguished from each other, such as when co-expressed in the same cell. In some embodiments, distinguishable fluorescent proteins, such as those with distinguishable excitation and/or emission spectra can be used to differentiate between the target polypeptide proteolytic products.

Any type of compound, such as the ADAM9 inhibitors described above, can be screened and identified using the methods described herein. The compounds screened can range from small organic molecules to large polymers and biopolymers, and can include, by way of example and not limitation, small organic compounds, saccharides, carbohydrates, polysaccharides, lectins, peptides and analogs thereof, polypeptides, proteins, antibodies, oligonucleotides, polynucleotides, nucleic acids, etc. In some embodiments, the candidate compounds screened are small organic molecules having a molecular weight in the range of about 100-2500 daltons. Such candidate molecules will often comprise cyclical structures composed of carbon atoms or mixtures of carbon atoms and one or more heteroatoms and/or aromatic, polyaromatic, heteroaromatic and/or polyaromatic structures. The candidate agents may include a wide variety of functional group substituents. In some embodiments, the substituent(s) are independently selected from the group of substituents known to interact with proteins, such as, for example, amine, carbonyl, hydroxyl and carboxyl groups.

The candidate compounds may be screened on a compound-by-compound basis or, alternatively, using one of the myriad library techniques commonly employed in the art. For example, synthetic combinatorial compound libraries, natural products libraries and/or peptide libraries may be screened using the assays of the invention to identify compounds that inhibit or reduce ADAM9 activity.

The polynucleotide sequence for preparing expression constructs for target polypeptides FGFR2iiib, EGF, CD40, EphB4, Flk1, Tie-2, VE-cadherin and VCAM can use information available to the skilled artisan. The nucleotide and amino acid sequences for the human forms of the target polypeptides are available as follows: FGFR2iiib: Swiss-Pro/Genbank Accession Nos. M97193. 1, NM_{—}000141.4, and NM_{—}022970.3;

EGF: Swiss-Pro/Genbank Accession Nos. P01133, NM_{—}001963.3, and NP_{—}001954.2;
CD40: Swiss-Pro/Genbank Accession Nos. P25942, NM_{—}001250.4, and NM_{—}152854.2;
EphB4: Swiss-Pro/Genbank Accession Nos. P54760 and NM_{—}004444.4;
Flk1/VEGFR2/KDR: Swiss-Pro/Genbank Accession Nos. No. P54760 and NM_{—}002253.2;
Tie-2: Swiss-Pro/Genbank Accession Nos. Q02763 and NM_{—}000459.3;
VE-cadherin: Swiss-Pro/Genbank Accession Nos. NM_{—}001795.3; and NM_{—}009868.4; and
VCAM: Swiss-Pro/Genbank Accession Nos. P19320, NP_{—}001069.1, and NP_{—}542413.1.

When co-expressed in cells for a cell-based assay system, virtually any type of cell in which the expressed ADAM9 polypeptide is active can be used, including, bacterial cells, yeast cells, insect cells, and animal cells, including mammalians cells. The selection of the appropriate cells will be within the skill of those in the art. Exemplary mammalian cells include, among others, CV-1/COS (e.g., Cos-7 cells), MDCK, CHO, NIH3T3, Rat-1, HeLa, HEK-293, LNCaP, MCF7, and various cultured tumor cell lines (e.g., A549, EBC-1, MV3 melanoma; see, e.g., Shintani et al., 2004, Cancer Research 64, 4190-4196). Other types of cells that can be used include cultured fibroblasts and endothelial cell lines. These and other cells are described in, among others, American Type Culture Collection (ATCC) and European Collection of Cell Cultures (ECACC).

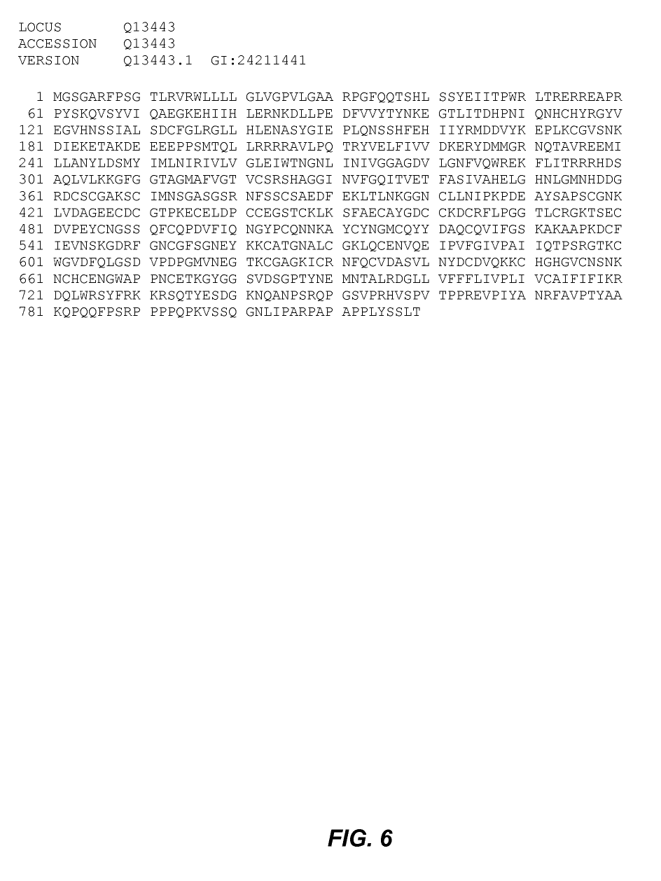

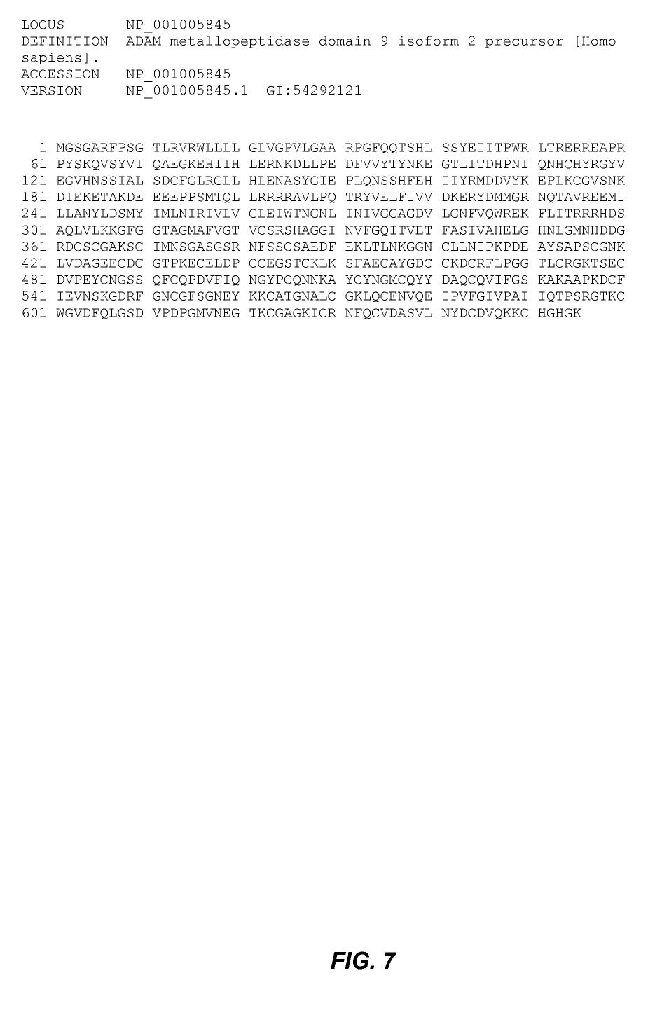

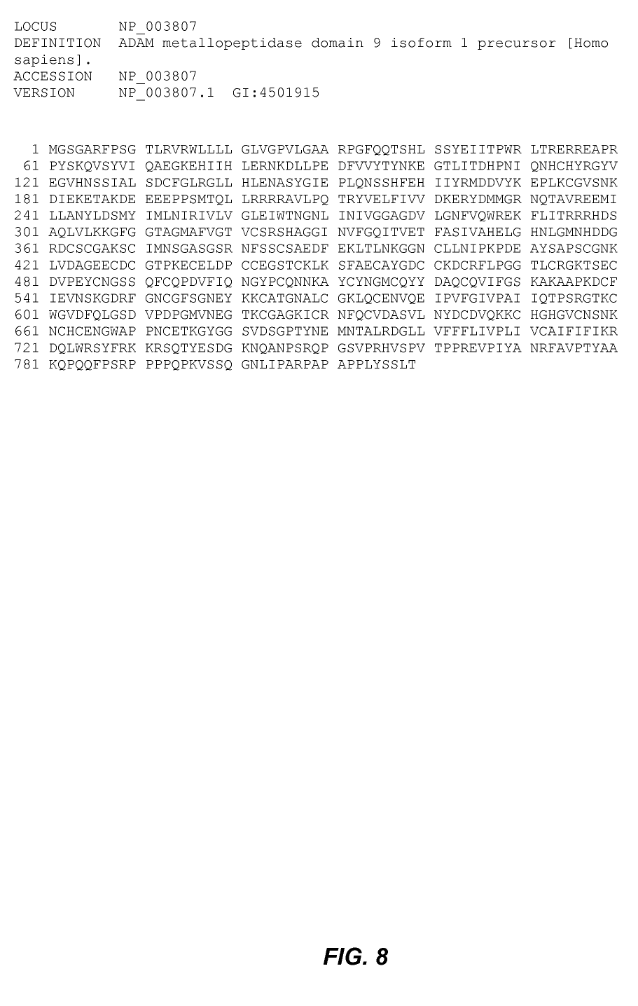

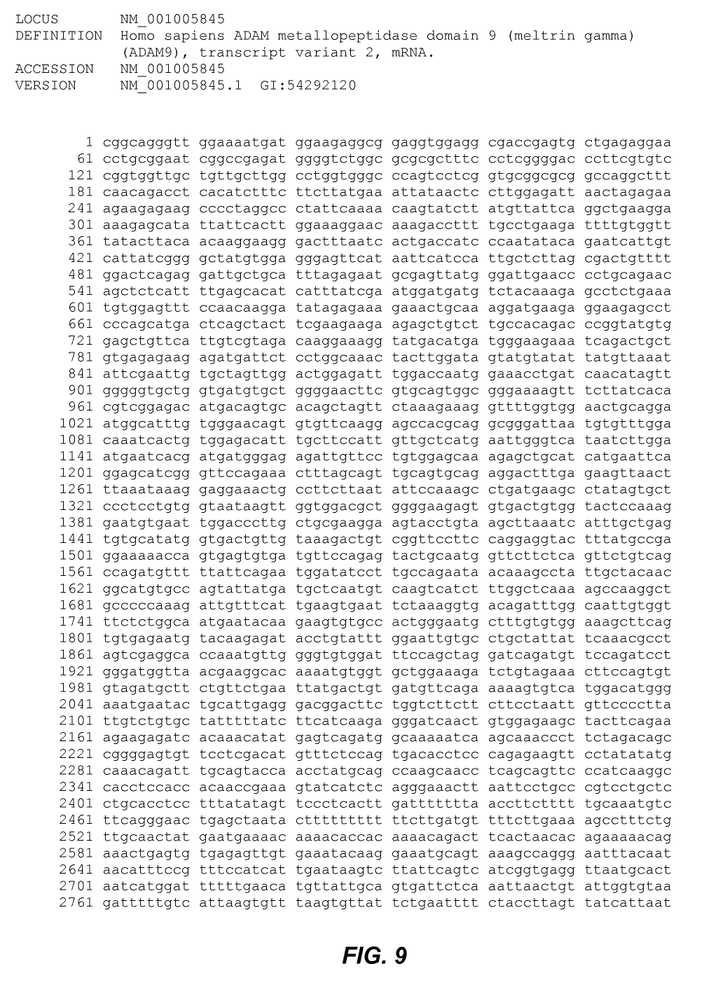

Expression vectors and cells expressing ADAM9 and target polypeptides can be made using routine techniques in the art, as provided in the Examples. The amino acid sequence of human ADAM9 is described in Weskamp et al., 1996, J. Cell Biol. 132(4):717-726, and Swiss-Pro/Genbank Accession No. Q 13443 (also shown in FIG. 6). Guidance for expressing polypeptides is also provided in reference works of Ausubel, supra, and Sambrook, supra. Methods of determining amounts of polypeptide expression gene products are well-known, and include by way of example and not limitation, antibody based detection, protein arrays and proteomics (Celis et al., 2000, FEBS. Lett. 480:2-16; Jungblat et al., 1999, Electrophoresis 20:2100-2110), and mass spectrometry (review in TO, 2000, Comb. Chem. High Throughput Science. 3:235-241; see also TTRAQ® product literature from Applied Biosystems). Reagents for carrying out these assays are either commercially available or can be designed from the ADAM9 polypeptide sequence illustrated in FIG. 6 using well-known, routine means. Anti-human ADAM9 antibody is described in, among others, US 2006/0172350 and Mahimkar et. al., 2005, J Am Soc Nephrol 11 :595-603.

### EXAMPLES

The working examples that follow, which are intended to be illustrative and not limiting, highlight various features of the invention(s) described herein.

For the examples that follow, all chemicals and reagents were purchased from Sigma, unless indicated otherwise. Isolectin B4 was purchased from Vector Labs (Burlingame, Calif.), the rabbit anti mouse NG2 antibody was from Chemicon International (Temecula, Calif.), and the -CD31 antibody was from BD Biosciences/Pharmingen (San Diego, Calif.). The anti-ADAM9 and ADAM 15 monoclonal antibodies used for immunofluorescence analysis and immunohistochemistry (Horiuchi et al., 2003. Mol. Cell Biol. 23:5614-5624; Weskamp et al., 2002, Mol. Cell. Biol. 22: 1537-1544.) and the polyclonal anti-ADAM9 antibody used for Western blot (Weskamp et al., Cell Biol. 132:717-726) have been previously described.

### Example 1

### ADAM9 in Oxygen Induced Retinopathy in ADAM9 −/− Knockout Mice

Oxygen Induced Retinopathy (OIR) mouse model. The response of wild-type and Adam9 −/− mice to relative hypoxia was assessed by using the oxygen-induced retinopathy model as described previously (Chen and Smith, 2007, Angiogenesis 10:133-40; Hammes et al., 1996, Nat Med 2:529-33; Horiuchi et al., 2003, Mol. Cell Biol. 23:5614-5624; Smith et al., 1994, Invest Ophthalmol Vis Sci 35:101-11). Since the animals were of mixed genetic background (129/SvJ and C57BL16J), initial experiments were performed by comparing wildtype and Adam9 −/− littermates that were offspring of matings of heterozygous Adam9 +/− parents. To corroborate the results from experiments with wildtype and Adam9 −/− littermates, and to increase the number of animals included in this analysis, litters consisting only of either Adam9 −/− or wildtype mice were generated by collecting either Adam9 −/− or wildtype males and females from one set of heterozygous Adam9 +/− parents, and then mating these to generate wildtype or Adam9 −/− litters. These wildtype and Adam9 −/− litters were closely related as they were derived from the same heterozygous grandparents.

For the OIR model, mice were exposed to an oxygen concentration of 75% from post-natal day 7 to 12 in a plexiglass chamber connected to an oxygen regulator, along with their nursing mother. At postnatal day 12 (P12), the animals were returned to normal room air. The resulting relative hypoxia triggers a proliferative response in the retinal vasculature (Chen et al., supra). Five days after the return to normoxic conditions (Le., P17), the animals were sacrificed. All experiments involving mice were approved by the Institutional Animal Care and Use Committees of the Hospital for Special Surgery.

Following euthanasia, both eyes were removed and fixed in 4% paraformaldehyde (PFA). The next day, one eye was processed for sectioning, and the second for a whole mount analysis of neovascularization. The eye that was intended for histological evaluation of neovascularization was embedded in paraffin, sectioned (6 μm thickness), and stained with hematoxylin and eosin. About 150 sections were prepared from each eye; and two to five sections on each side of the optic nerve, 30 to 90 μm apart, were used for quantitation of neovascularization in a double blinded manner by counting endothelial cell nuclei on the vitreal side of the internal limiting membrane, as previously described (Horiuchi et al., supra). The number of endothelial nuclei per section was averaged for each eye, and the unpaired Student t test (equal variation, two sided) was used to statistically evaluate the difference between Adam9 −/− and wild-type mice.

The second eye from each animal was used to evaluate the size of the central avascular area that develops in the retinas of mice that were subjected to the OIR model. Following overnight fixation with 4% PFA, the eyes were washed five times with PBS and incubated overnight in LBB (lectin blocking buffer 1% BSA, 0.1% Triton X-100, 0.1 M glycine, in PBS). Then the retinas were excised, flat mounted on microscope slides and incubated with LBB for an additional 2 hrs. Fluorescein labeled isolectin B4 was diluted 1:200 in 0.2× LBB and then added to the retinas and incubated overnight at 4° C. After washing, a coverslip was applied, and whole mounted samples were photographed using a Qimaging Retiga EXi camera attached to a Nikon Eclipse E600 fluorescent microscope. The size of the avascular area and of the total retina were outlined in Photoshop, and the percentage of the avascular area compared to the total retina was calculated using NIH Image software.

Immunohistochemistrv and immunofluorescence analysis. Frozen and paraffin embedded samples were used for immunostaining studies. Eyes and tumors were fixed in fresh 4% PFA overnight at 4° C. The fixed specimens were washed three times with PBS, then stored in 70% ethanol or immediately processed for paraffin embedding. For frozen sections, the PBS washed samples were immersed in 20% sucrose overnight at 4° C., and then frozen in Tissue-Tek O.C.T. compound (Sakura Finetek, Torrance, Calif.) mixed with 20% sucrose in a 1:1 ratio. Sections (10 μm) were cut, mounted on slides and postfixed with ice-cold acetone for 10 min. Both frozen and paraffin sections were immersed in 0.3% and 3% H₂O₂ respectively to inactivate the endogenous peroxidase, preincubated with 10% normal goat serum/2% bovine serum albumin/PBS for 1 h, and then incubated for 1 h with anti ADAM9 or ADAM15EC-Fc antiserum or platelet endothelial cell adhesion molecule-1 (PECAM1/CD31) antibodies (Horiuchi et al., supra). Bound antibodies were detected with biotin-conjugated goat anti-rabbit IgG followed by coupled to Avidin according to the manufacturer's instructions (Vector Labs). After development of the color reaction with diaminobenzidine solution (Vector Labs), the sections were counterstained with hematoxylin.

Flat mounted retinas were incubated with modified LBB (0.5% Triton X-100) for 4 h, washed three times with PBS and incubated with either NG2 or an anti-ADAM9 monoclonal antibody (Weskamp et al., supra) overnight at 4° C. Retinas were washed three times with PBS and secondary anti-rabbit TRITC or FITC conjugated antibodies were added. After 2 h incubation, retinas were washed three times with PBS and stained overnight with FITC or TRITC isolectin.

In addition to whole retina staining, 6 μm sections of paraffin embedded eyes were analyzed by histochemistry and immunofluorescence. Eye sections were deparaffinized, rehydrated through a graded alcohol series and heated in 10 mM sodium citrate for antigen retrieval. Samples were treated as above for biotin/avidin HRP-staining or FITC conjugated secondary antibodies against the primary ADAM9 monoclonal antibody.

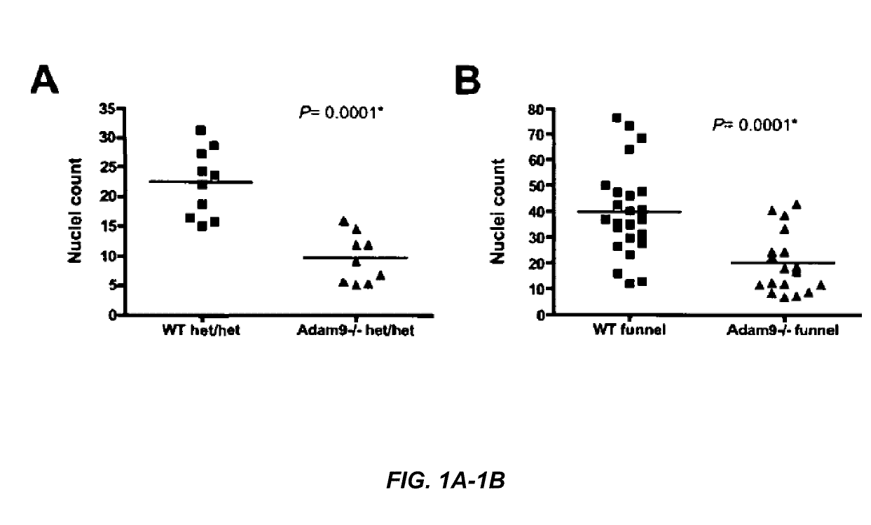

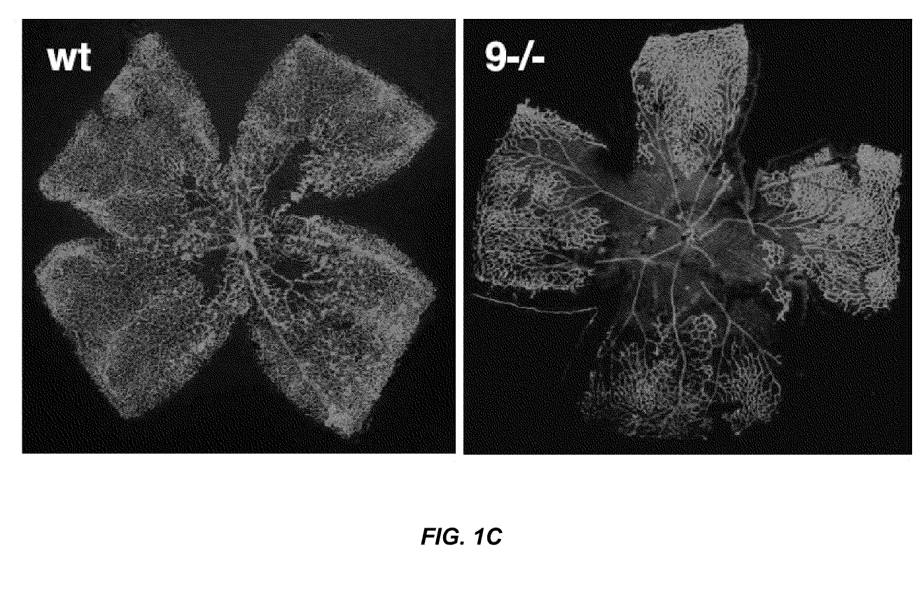

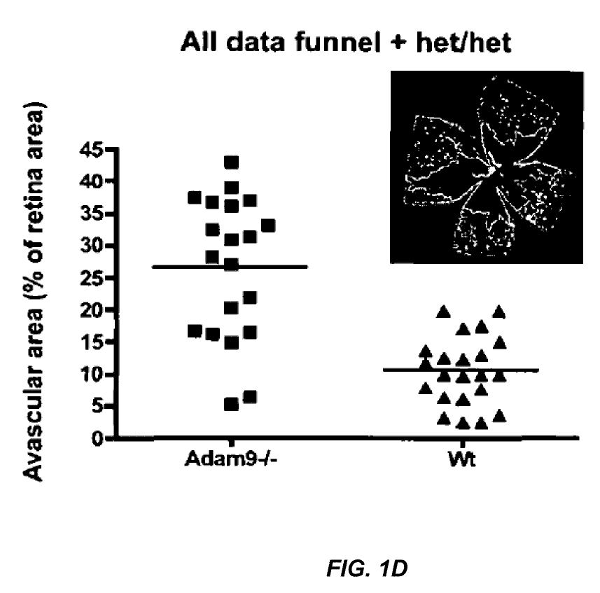

Results. To address a potential role of ADAM9 in pathological retinal neovascularization, Adam9 −/— mice and wild type littermate controls were tested in a mouse model for oxygen induced retinopathy (OIR). As shown in FIG. 1A, there was a significant reduction in number of vascular cell nuclei that contributed to pathological neovascularization in Adam9 −/— mice compared to wild type littermate controls (n wt=10; n ko=9; p<0.0001). To corroborate these results with increased numbers of mice, highly related litters consisting only of Adam9 −/− or wild type mice were subjected to the OIR model. The results also showed a significant reduction of pathological neovascularization in Adam9 −/− mice compared to wildtype controls (p<0.0001). Moreover, an analysis of the avascular area and the formation of neovascular tufts in whole-mount preparations of retina stained with FITC labeled lectin, which labels endothelial cells, showed that Adam9 −/− mice have a larger central avascular area and strongly reduced tuft formation compared to wild type controls (FIG. 1C). On average, the ratio of the avascular area to the whole retina was significantly larger in Adam9 −/− mice (27%), than in wildtype controls (10%). Taken together, these finding indicate that ADAM9 has a detrimental role during oxygen-induced retinopathy in mice.

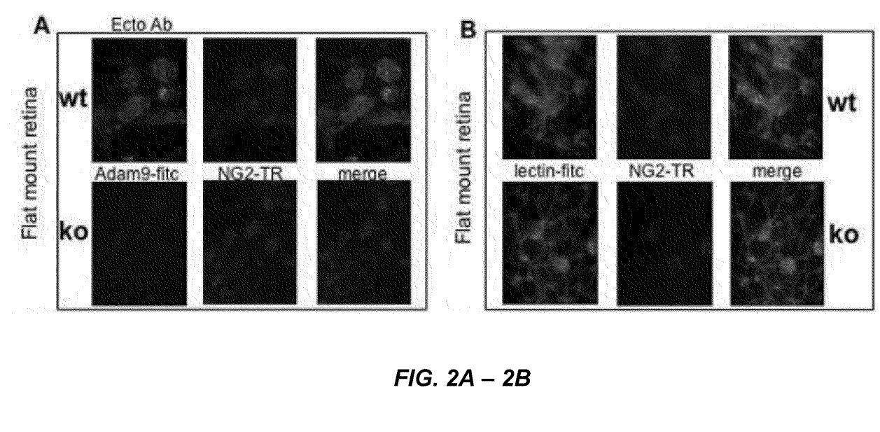

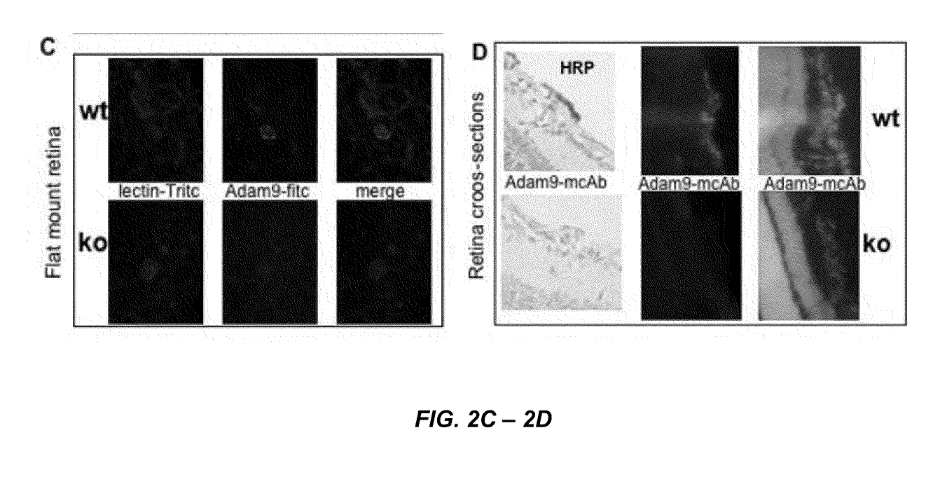

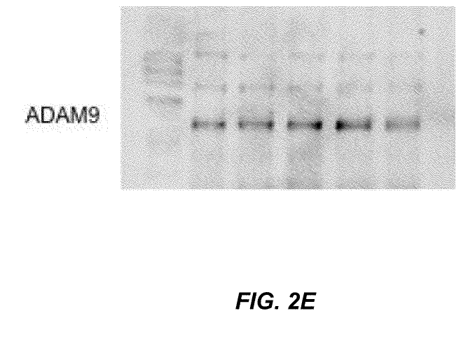

To determine whether the expression of ADAM9 in the retina changes during the course of an OIR experiment, a Western blot analysis were performed on extracts of retinas from animals that were prepared 1 to 5 days after their return to room air (FIG. 2E). The amounts of ADAM9 protein detected in different samples did not change significantly. Since a Western blot of whole retinas might not uncover local changes in ADAM9 expression, whole-mount preparations of retinas of wildtype mice subjected to the OIR model were also stained with a mouse monoclonal antibody against ADAM9 (detected with an FITC labeled secondary antibody and with lectin (TRITC labeled) to label endothelial cells. Immunofluorescence microscopy of these samples showed that ADAM9 is very highly expressed in neovascular tufts following the OIR model, whereas no evident increase in ADAM9 expression was seen in other TRITC-lectin labeled endothelial cells or in other parts of the retina. When identical experiments were performed with retinas from Adam9 −/− mice subjected to the OIR models, the anti-ADAM9 antibody did not label neovascular tufts that could be clearly visualized with TRITC-lectin, demonstrating that the staining observed in wildtype retinas was specific for ADAM9. A comparison of the expression of ADAM9 in neovascular tufts in wild type mice with that of the pericyte marker NG2 (see FIG. 2A and 2B) showed little, if any, overlap, suggesting that ADAM9 expressing cells are distinct from the NG2 expressing pericytes. These results also indicate that the pericytes in neovascular tufts are intermingled with endothelial cells and ADAM9 expressing cells, whereas pericytes surround endothelial cells in a typically organized capillary. The high expression of ADAM9 in neovascular tufts was also corroborated in an HRP stained section of a retina taken from a wildtype animals as well as by immunofluorescence analysis. Sections of a tuft from an Adam9 −/− mouse served as a control for the specificity of the anti-ADAM9 antibody used here in immunohistochemistry and immunofluorescence analysis. Finally, antibodies against ADAM15, which is also highly expressed in neovascular tufts (Horiuchi et al., supra), stained the tufts in both wildtype and Adam9 −/− samples (FIG. 1E).

### Example 2

### ADAM9 and Effect on Melanoma Cell Growth

Heterotopic injection of B16FO mouse melanoma cells. Age- and sex matched litters of wild-type and Adam9 −/− mice, derived from the same Adam9 +/grandparents as described above, were injected subcutaneously with 1×10⁶ B16F0 mouse melanoma cells resuspended in PBS. The animals were sacrificed 2 to 3 weeks after injection, depending on the severity of the tumor burden, and the tumors were removed and weighed.

For quantitation and comparison of individual trials, the average weight of tumors from wild-type controls in a given experiment was used as a reference to calculate the weight of each tumor as a percentage of the wild-type average. The unpaired Student t test was used for statistical evaluation. Immunofluorescence was further performed to evaluate the vascularity of the tumor specimens. The frozen sections were prepared as described above and incubated with anti-PECAM/CD31 antibody. Bound antibodies were detected by Cy3-conjugated AffiniPure donkey anti-rat IgG purchased from Jackson ImmunoResearch, West Grove, Pa.). Quantitative analysis was performed using Scion Image (beta 4.0.2).

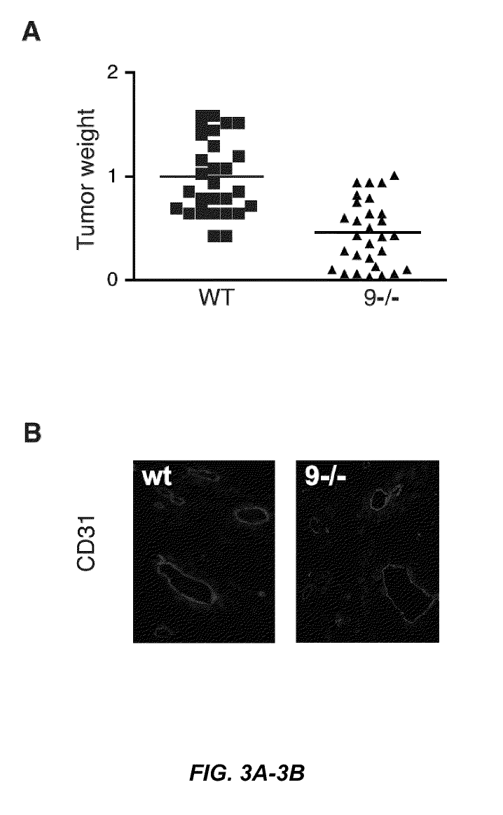

Results This second approach to evaluate pathological neovascularization in mice monitors the growth of heterotopically injected tumor cells in wildtype and knockout mice, which can also provide insights into the contribution of host derived cells and factors to tumorigenesis. To minimize potential effects of the mouse genetic background on this model, tumor growth in litters of wildtype mice or Adam9 −/− mice that had been bred from offspring of the same heterozygous parents (see above, and materials and methods for details) were compared. In three separate experiments, the average weight of tumors that developed after subcutaneous injection of 1×10⁶ B16F0 cells in Adam9 −/− mice was significantly reduced compared to wildtype controls that had been injected with the same culture of cells at the same time (FIG. 3A, reduction in tumor weight in Adam9 −/versus wildtype controls=60%).

### Example 3

### Assays for ADAM9 Activity Using Cell-Based Ectodomain Shedding Assays

Construction of expression plasmids and cell-based protein ectodomain shedding assays. To generate expression plasmids for alkaline phosphatase tagged receptors with role in angiogenesis, cDNAs for murine VE-Cadherin, Tie2, CD40, EphB4, EphrinB2, CD34, VCAM-1, ICAM-1 and E-selectin were obtained from the ATCC. The cDNA for Flk1 was the kind gift of Urban Deutsch (Univ. Berne, Switzerland). All AP-tagged fusion proteins were designed to include at least the first extracellular domain, the transmembrane domain and the cytoplasmic tail of the target substrate. Corresponding primers for each gene were used to generate cDNA by PCR amplification using the appropriate full length cDNA as template and ligated into the pAPtag5 expression plasmid (Genehunter, Nashville, Tenn.). The following primers were used to generate PCR products corresponding to the carboxyl end of each open reading frame, using the full-length cDNA as template:

| | |
|---|---|
| FLK-1 (amino acids 605-859): | |
| 5-GATGCTCTTTGGAAACTGAATGGCACCA-3; | (SEQ ID NO: 6) |
| 5-AGTCGCTGTCTTGTCAATTCCAAAAGCGT-3; | (SEQ ID NO: 7) |
| E-Selectin (amino acids 31-612): | |
| 5-ATGACGTATGATGAAGCCAGTGCA-3; | (SEQ ID NO: 8) |
| 5-GTTCCTGATTGTTTTGAACCTAGA-3; | (SEQ ID NO: 9) |
| CD34 (amino acids 31-385): | |
| 5-ATGAGTCTTGACAACAACGGTA-3; | (SEQ ID NO: 10) |
| 5-TCACAATTCGGTATCAGCCACCACG-3; | (SEQ ID NO: 11) |
| CD40 (amino acids 24-278): | |
| 5-ACTGCATGCAGAGAAAAACAGTA-3; | (SEQ ID NO: 12) |
| 5-TCACTGTCTCTCCTGCACTGA-3; | (SEQ ID NO: 13) |
| ICAM-1 (amino acids 26-532): | |
| 5-AATG CCCAGACATCTGTGTCCCCC-3; | (SEQ ID NO: 14) |
| 5-TCAGGGAGGCGTGGCTTGTG-3; | (SEQ ID NO: 15) |
| VCAM-1 (amino acids 28-739): | |
| 5-GAGATCTCCCCTGAATACA-3; | (SEQ ID NO: 16) |
| 5-CTACACTTTGGATTTCTGTGC-3; | (SEQ ID NO: 17) |
| Tie-2 (amino acids 580-833): | |
| 5-TCCCTGCAAACAACAAGTGATCA-3; | (SEQ ID NO: 18) |
| 5-AAAGTTGCCCTCTCCGATCACG-3. | (SEQ ID NO: 19) |

Each amplified cDNA was cloned in frame with human alkaline phosphatase into pAP-tag (Genehunter, Nashville, Tenn.), and all constructs were sequenced to rule out undesired mutations.

Stimulation of ADAM9-dependent shedding. Expression constructs for ADAM9 and the catalytically inactive E>A mutant were described previously (Roghani et al., 1999, J. BioI. Chem. 274:3531-3540.). Alkaline phosphatase shedding assays were performed as described (Weskamp et al., 1996, Cell Biol. 132:717-726; Zheng et al., 2002, J. BioI. Chem. 277:42463-42470) in (mouse embryonic fibroblasts) MEF cells and in pig aortic endothelial cells (PAE cells, kindly provided by Dr. Shahin Rafii). Reactive oxygen species (ROS) assays were performed using a modified version of the procedure reported by Sung et al., 2006, Cancer Res 66:9519-26). Specifically, 20 hours post-transfection, cells maintained at 50-60% culture density were washed twice with OptiMem over the course of 1 hour. Cells were then cultured in OptiMem supplemented with peroxide for 5 hours at various concentrations, as indicated. The resulting supernatants and lysates were collected and assayed in triplicate for alkaline phosphatase activity as described previously (Sahin et al., 2006, “A sensitive method to monitor ectodomain shedding of ligands of the epidermal growth factor receptor,” In Epidermal Growth Factor: Methods and Protocols, In P. J. B. and T. B. Patel eds., Vol. 327, p. 99-113., Humana Press Inc., Totowa, N.J.; Sahin et al., J. Cell Biol. 164:769779; Zheng et al., 2002, J. BioI. Chem. 277:42463-42470). All data presented here representative example from at least three independent assays.

For ADAM9 immunoblot analysis, peroxide-treated lysates were clarified by centrifugation and collected on Con A-Sepharose 48 (GE Healthcare), boiled in SDS-PAGE sample buffer under reducing conditions and subjected to SDS-PAGE and immunoblotting (Weskamp et al., 1996, J. Cell Biol. 132:717-726).

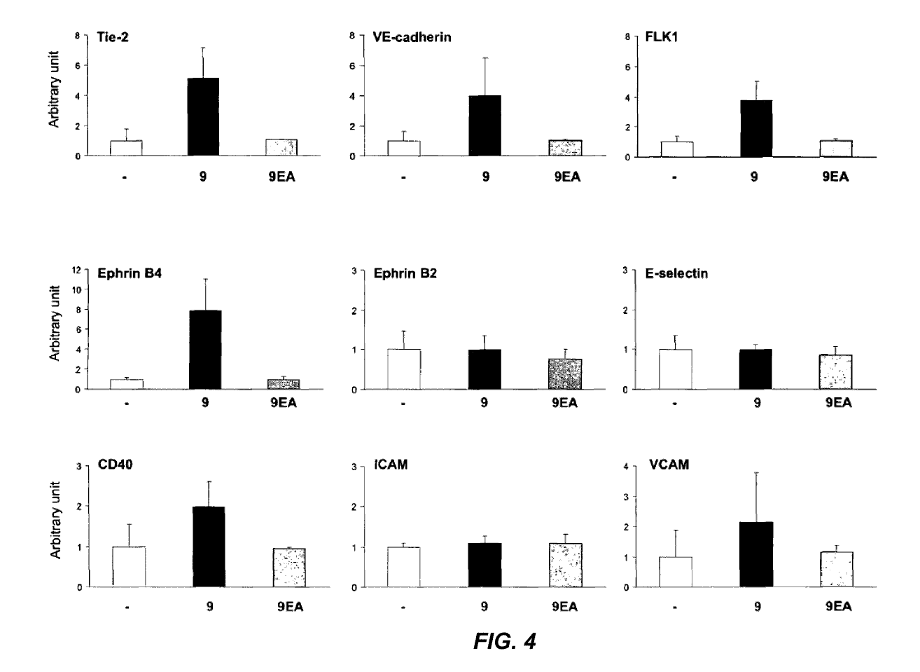

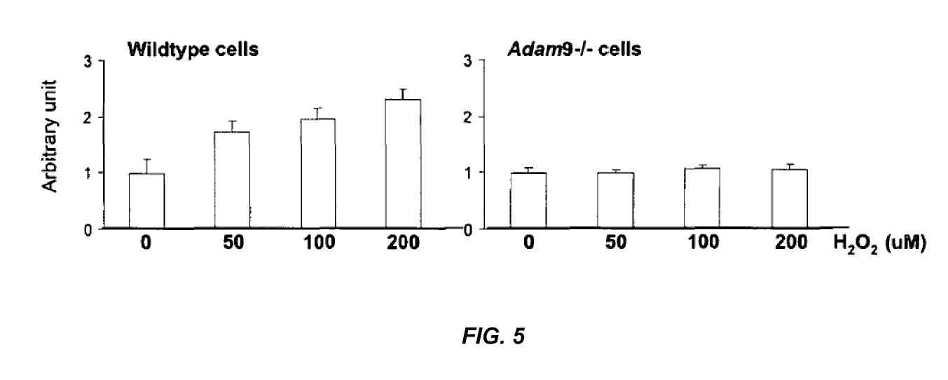

Shedding of ADAM9 Target Polypeptides. The high expression of ADAM9 in endothelial cells in vascular tufts of mice subjected to the OIR model as well as the decreased pathological retinal neovascularization and the reduced growth of heterotopic B 16F0 tumors in Adam9 −/− mice raised the possibility that ADAM9 might have important functions in endothelial cells. Since ADAM9 is known to function as a membrane anchored protease, and because proteolysis can posttranslationally regulate the function of several membrane proteins, an assay was developed to examine if ADAM9 could affect pathological neovascularization by processing membrane proteins with roles in angiogenesis and neovascularization. To address this possibility, ADAM9 was expressed in Cos-7 cells together with several membrane anchored receptors with critical functions in angiogenesis (CD40, EprinB2, EphB4. E-selectin, Flk-1, ICAM-1, VACM, Tie-2. VE-cadherin, Flk-1, CD34 and CD36). Each of these candidate substrates contained an alkaline phosphatase tag in its ectodomain to facilitate detection of the released protein in the culture supernatant (Sahin et al., supra). In this context it should be noted that addition of an alkaline phosphatase tag does not affect the release of a number of other membrane proteins, such as TGFα or TNF, and is therefore used here as a sensitive reporter for shedding of these membrane proteins. In co-expression experiments with ADAM9 and candidate substrates, the control experiment consisted of cotransfection of the substrates with the inactive ADAM9 E>A catalytic site mutant to monitor baseline shedding by endogenous sheddases in Cos-7 cells. As shown in FIG. 4, overexpression of ADAM9 increased the shedding of CD40, EphB4, Flk1, Tie-2, VE-cadherin and VCAM compared to cells overexpressing ADAM9 E>A together with these substrates. Overexpression of ADAM9 did not increase the shedding of E-selectin, ICAM-1, Ephrin B2, CD34 and CD36 compared to ADAM9 E>A, at least under the conditions used here. These results demonstrate that ADAM9 is capable of processing several proteins with established functions in angiogenesis and neovascularization in gain of function experiments in which it is overexpressed.

Regulation of ADAM9-dependent shedding by reactive oxygen species. The ability of overexpressed ADAM9 to cleave several substrate proteins in “gain of function” experiments raised questions about the requirement for ADAM9 for the shedding of these substrates in “loss of function” experiments with cells lacking ADAM9. This might be most evident under conditions in which the expression of endogenous ADAM9 is upregulated. Since the expression of ADAM9 can be increased by treatment of prostate cancer cells with reactive oxygen species (ROS) (Sung et al., 2006, Cancer Res 66:9519-26), the effect of ROS treatment on the expression of ADAM9 in mouse embryonic fibroblasts was examined. When MEF cells from wildtype mice were treated with increasing concentrations of H₂O₂ for 6 hrs, an increase in ADAM9 expression was observed in cells treated with the highest concentration of H₂O₂ (200 μM).

To assess whether this increase in expression of ADAM9 also increased ADAM9-dependent proteolysis, EphB4, previously established as a substrate for overexpressed ADAM9 in Cos-7 cells, was expressed in embryonic fibroblasts isolated from Adam9 −/− mice or wildtype controls. Treatment of wt MEF cells with 100 or 200 μM H₂O₂ for 6 hrs resulted in a strong increase in the shedding of transiently expressed EphB4 (FIG. 4), whereas no increase in EphB4 shedding was seen when Adam9 −/− cells expressing EphB4 were treated with 100 or 200 μM H₂O₂. The shedding of EphB4 from Adam9 −/− cells could be rescued by transfection with ADAM9, however, the resulting activity could not be further enhanced by treatment with H₂O₂. These results suggest that the increased in ADAM9-dependent processing of EphB4 in wt MEF cells following treatment with H₂O₂ depends on activation of the expression of endogenous ADAM9, and not on posttranslational activation of overexpressed ADAM9, as the plasmid used to express ADAM9 does not contain the endogenous promoter region with putative ROS response elements. Taken together, these “loss-of-function” results clearly corroborate that upregulation of endogenous ADAM9, in this case via treatment with H₂O₂, results in increased ADAM9 activity, as evidenced by enhanced ADAM9 dependent shedding of EphB4.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A method of treating pathological retinal neovascularization, comprising administering to an eye of a patient in need of treatment for pathological retinal neovascularization an amount of an ADAM9 inhibitory compound effective to reduce pathological retinal neovascularization.

2. The method of claim 1 wherein the pathological retinal neovascularization in need of treatment is caused by diabetic retinopathy.

3. The method of claim 1 wherein the pathological retinal neovascularization in need of treatment is caused by macular degeneration.

4. The method of claim 1 wherein the pathological retinal neovascularization in need of treatment is caused by retinopathy of immaturity.

5. The method of claim 1 in which the ADAM9 inhibitory compound inhibits an activity of ADAM9.

6. The method of claim 5, wherein the activity that is inhibited is the ADAM9 metalloproteinase activity.

7. The method of claim 5, wherein the activity that is inhibited is the ADAM9 disintegrin activity or in cell-cell or cell matrix interactions.

8. The method of claim 5 in which the ADAM9 inhibitory compound is selected from the group consisting of an antisense oligonucleotide, an siRNA, an miRNA, a small organic molecule, an enzyme, an antibody, a peptide, a hormone, and a polynucleotide encoding a polypeptide.

9. The method of claim 1 in which the ADAM9 inhibitory compound inhibits transcription of a gene encoding ADAM9.

10. The method of claim 1 in which the ADAM9 inhibitory compound is a polynucleotide or oligonucleotide comprising a sequence complementary to a region of the ADAM9 gene.

11. The method of claim 1 in which the ADAM9 inhibitory compound inhibits translation of an mRNA encoding ADAM9.

12. The method of claim 11 in which the ADAM9 inhibitory compound is an antisense oligonucleotide, an iRNA, an siRNA, or a nucleic acid encoding an antisense oligonucleotide, an iRNA or an siRNA.

13. The method of claim 1 in which the ADAM9 inhibitory compound promotes the degradation of ADAM9 protein.

14. The method of claim 1 in which the ADAM9 inhibitory compound is selected from the group consisting of an antibody, an antibody fragment, an Fab fragment, an Fab′ fragment, an F(ab′)₂ fragment, an Fv fragment, a linear antibody, a humanized antibody, a monoclonal antibody, a chimeric antibody, a single chain antibody, a diabody, an aptamer and an isolated complementarity determining region fused to another molecule, wherein said ADAM9 inhibitory compound has binding specificity for ADAM9 protein.

15. The method of claim 1 in which the ADAM9 inhibitory compound is an antibody, or fragment thereof, conjugated to a moiety selected from the group consisting of a toxin, a radioactive isotope, a neutron-capture reagent, and a fluorochrome.

16. The method of claim 1 in which the ADAM9 inhibitory compound is administered intravitreously.

17. The method of claim 1 in which the ADAM9 inhibitory compound is administered to the surface of the eye.

18. A method of inhibiting angiogenesis, comprising administering to a patient in need thereof an amount of an ADAM9 inhibitory compound effective to reduce angiogenesis or neovascularization in said patient.

19. The method of claim 18, wherein the angiogenesis is in a tumor and the ADAM9 inhibitory compound is administered directly into the tumor.

20. A method of treating rheumatoid arthritis, comprising administering to a patient in need of treatment for rheumatoid arthritis an amount of an ADAM9 inhibitory compound effective to reduce neovascularization of a joint of said patient.

21. The method of claim 20, wherein said ADAM9 inhibitory compound is administered directly into said joint.

22. A method of screening for an agent that inhibits ADAM9 mediated angiogenesis, comprising contacting ADAM9 polypeptide with a target polypeptide in presence of a candidate agent, and determining the presence of proteolyzed target polypeptide, wherein the target polypeptide comprises at least one or more of polypeptides CD40, EphB4, Flk1, Tie-2, VE-cadherin and VCAM.

23. The method of claim 22 in which the target polypeptides includes at least EphB4.

24. The method of claim 22 in which the target polypeptide includes in addition to one or more of CD40, EphB4, Flk1, Tie-2, VE-cadherin and VCAM, one or more of FGFR2iiib and EGF.

25. The method of claim 22 in which ADAM9 and the target polypeptides are co-expressed in a cell and the cell is incubated with the candidate agent.

26. The method of claim 25 in which the proteolyzed target polypeptide released from the cell is detected.

27. The method of claim 22 in which a detectable moiety is bound to the target polypeptide.

28. The method of claim 27 in which the detectable moiety comprises a fluorescent moiety, an antibody epitope tag, a reporter enzyme, or a fluorescent protein.

29. The method of claim 27 in which the detectable moiety bound to each type of angiogenic polypeptide are distinguishable.

30. The method of claim 22 in which the candidate compound is a small organic molecule


SEQUENCE LISTING (95 sequences)

SEQ ID NO: 1 - protein, 819 aa, Homo sapiens
MGSGARFPSGTLRVRWLLLLGLVGPVLGAARPGFQQTSHLSSYEIITPWRLTRERREAPRPYSKQVSYVIQAEGKEHIIHLERNKDLLPEDFVVYTYNKEGTLITDHPNIQNHCHYRGYVEGVHNSSIALSDCFGLRGLLHLENASYGIEPLQNSSHFEHIIYRMDDVYKEPLKCGVSNKDIEKETAKDEEEEPPSMTQLLRRRRAVLPQTRYVELFIVVDKERYDMMGRNQTAVREEMILLANYLDSMYIMLNIRIVLVGLEIWTNGNLINIVGGAGDVLGNFVQWREKFLITRRRHDSAQLVLKKGFGGTAGMAFVGTVCSRSHAGGINVFGQITVETFASIVAHELGHNLGMNHDDGRDCSCGAKSCIMNSGASGSRNFSSCSAEDFEKLTLNKGGNCLLNIPKPDEAYSAPSCGNKLVDAGEECDCGTPKECELDPCCEGSTCKLKSFAECAYGDCCKDCRFLPGGTLCRGKTSECDVPEYCNGSSQFCQPDVFIQNGYPCQNNKAYCYNGMCQYYDAQCQVIFGSKAKAAPKDCFIEVNSKGDRFGNCGFSGNEYKKCATGNALCGKLQCENVQEIPVFGIVPAIIQTPSRGTKCWGVDFQLGSDVPDPGMVNEGTKCGAGKICRNFQCVDASVLNYDCDVQKKCHGHGVCNSNKNCHCENGWAPPNCETKGYGGSVDSGPTYNEMNTALRDGLLVFFFLIVPLIVCAIFIFIKRDQLWRSYFRKKRSQTYESDGKNQANPSRQPGSVPRHVSPVTPPREVPIYANRFAVPTYAAKQPQQFPSRPPPPQPKVSSQGNLIPARPAPAPPLYSSLT

SEQ ID NO: 2 - protein, 655 aa, Homo sapiens
MGSGARFPSGTLRVRWLLLLGLVGPVLGAARPGFQQTSHLSSYEIITPWRLTRERREAPRPYSKQVSYVIQAEGKEHIIHLERNKDLLPEDFVVYTYNKEGTLITDHPNIQNHCHYRGYVEGVHNSSIALSDCFGLRGLLHLENASYGIEPLQNSSHFEHIIYRMDDVYKEPLKCGVSNKDIEKETAKDEEEEPPSMTQLLRRRRAVLPQTRYVELFIVVDKERYDMMGRNQTAVREEMILLANYLDSMYIMLNIRIVLVGLEIWTNGNLINIVGGAGDVLGNFVQWREKFLITRRRHDSAQLVLKKGFGGTAGMAFVGTVCSRSHAGGINVFGQITVETFASIVAHELGHNLGMNHDDGRDCSCGAKSCIMNSGASGSRNFSSCSAEDFEKLTLNKGGNCLLNIPKPDEAYSAPSCGNKLVDAGEECDCGTPKECELDPCCEGSTCKLKSFAECAYGDCCKDCRFLPGGTLCRGKTSECDVPEYCNGSSQFCQPDVFIQNGYPCQNNKAYCYNGMCQYYDAQCQVIFGSKAKAAPKDCFIEVNSKGDRFGNCGFSGNEYKKCATGNALCGKLQCENVQEIPVFGIVPAIIQTPSRGTKCWGVDFQLGSDVPDPGMVNEGTKCGAGKICRNFQCVDASVLNYDCDVQKKCHGHGK

SEQ ID NO: 3 - protein, 819 aa, Homo sapiens; identical to SEQ ID NO: 1

SEQ ID NO: 4 - DNA, 4005 bp, Homo sapiens
cggcagggttggaaaatgatggaagaggcggaggtggaggcgaccgagtgctgagaggaacctgcggaatcggccgagatggggtctggcgcgcgctttccctcggggacccttcgtgtccggtggttgctgttgcttggcctggtgggcccagtcctcggtgcggcgcggccaggctttcaacagacctcacatctttcttcttatgaaattataactccttggagattaactagagaaagaagagaagcccctaggccctattcaaaacaagtatcttatgttattcaggctgaaggaaaagagcatattattcacttggaaaggaacaaagaccttttgcctgaagattttgtggtttatacttacaacaaggaagggactttaatcactgaccatcccaatatacagaatcattgtcattatcggggctatgtggagggagttcataattcatccattgctcttagcgactgttttggactcagaggattgctgcatttagagaatgcgagttatgggattgaacccctgcagaacagctctcattttgagcacatcatttatcgaatggatgatgtctacaaagagcctctgaaatgtggagtttccaacaaggatatagagaaagaaactgcaaaggatgaagaggaagagcctcccagcatgactcagctacttcgaagaagaagagctgtcttgccacagacccggtatgtggagctgttcattgtcgtagacaaggaaaggtatgacatgatgggaagaaatcagactgctgtgagagaagagatgattctcctggcaaactacttggatagtatgtatattatgttaaatattcgaattgtgctagttggactggagatttggaccaatggaaacctgatcaacatagttgggggtgctggtgatgtgctggggaacttcgtgcagtggcgggaaaagtttcttatcacacgtcggagacatgacagtgcacagctagttctaaagaaaggttttggtggaactgcaggaatggcatttgtgggaacagtgtgttcaaggagccacgcaggcgggattaatgtgtttggacaaatcactgtggagacatttgcttccattgttgctcatgaattgggtcataatcttggaatgaatcacgatgatgggagagattgttcctgtggagcaaagagctgcatcatgaattcaggagcatcgggttccagaaactttagcagttgcagtgcagaggactttgagaagttaactttaaataaaggaggaaactgccttcttaatattccaaagcctgatgaagcctatagtgctccctcctgtggtaataagttggtggacgctggggaagagtgtgactgtggtactccaaaggaatgtgaattggacccttgctgcgaaggaagtacctgtaagcttaaatcatttgctgagtgtgcatatggtgactgttgtaaagactgtcggttccttccaggaggtactttatgccgaggaaaaaccagtgagtgtgatgttccagagtactgcaatggttcttctcagttctgtcagccagatgtttttattcagaatggatatccttgccagaataacaaagcctattgctacaacggcatgtgccagtattatgatgctcaatgtcaagtcatctttggctcaaaagccaaggctgcccccaaagattgtttcattgaagtgaattctaaaggtgacagatttggcaattgtggtttctctggcaatgaatacaagaagtgtgccactgggaatgctttgtgtggaaagcttcagtgtgagaatgtacaagagatacctgtatttggaattgtgcctgctattattcaaacgcctagtcgaggcaccaaatgttggggtgtggatttccagctaggatcagatgttccagatcctgggatggttaacgaaggcacaaaatgtggtgctggaaagatctgtagaaacttccagtgtgtagatgcttctgttctgaattatgactgtgatgttcagaaaaagtgtcatggacatgggaaatgaatactgcattgagggacggacttctggtcttcttcttcctaattgttccccttattgtctgtgctatttttatcttcatcaagagggatcaactgtggagaagctacttcagaaagaagagatcacaaacatatgagtcagatggcaaaaatcaagcaaacccttctagacagccggggagtgttcctcgacatgtttctccagtgacacctcccagagaagttcctatatatgcaaacagatttgcagtaccaacctatgcagccaagcaacctcagcagttcccatcaaggccacctccaccacaaccgaaagtatcatctcagggaaacttaattcctgcccgtcctgctcctgcacctcctttatatagttccctcacttgatttttttaaccttctttttgcaaatgtcttcagggaactgagctaatactttttttttttcttgatgttttcttgaaaagcctttctgttgcaactatgaatgaaaacaaaacaccacaaaacagacttcactaacacagaaaaacagaaactgagtgtgagagttgtgaaatacaaggaaatgcagtaaagccagggaatttacaataacatttccgtttccatcattgaataagtcttattcagtcatcggtgaggttaatgcactaatcatggattttttgaacatgttattgcagtgattctcaaattaactgtattggtgtaagatttttgtcattaagtgtttaagtgttattctgaattttctaccttagttatcattaatgtagttcctcattgaacatgtgataatctaatacctgtgaaaactgactaatcagctgccaataatatctaatatttttcatcatgcacgaattaataatcatcatactctagaatcttgtctgtcactcactacatgaataagcaaatattgtcttcaaaagaatgcacaagaaccacaattaagatgtcatattattttgaaagtacaaaatatactaaaagagtgtgtgtgtattcacgcagttactcgcttccatttttatgacctttcaactataggtaataactcttagagaaattaatttaatattagaatttctattatgaatcatgtgaaagcatgacattcgttcacaatagcactattttaaataaattataagctttaaggtacgaagtatttaatagatctaatcaaatatgttgattcatggctataataaagcaggagcaattataaaatcttcaatcaattgaacttttacaaaaccacttgagaatttcatgagcactttaaaatctgaactttcaaagcttgctattaaatcatttagaatgtttacatttactaaggtgtgctgggtcatgtaaaatattagacactaatattttcatagaaattaggctggagaaagaaggaagaaatggttttcttaaatacctacaaaaaagttactgtggtatctatgagttatcatcttagctgtgttaaaaatgaatttttactatggcagatatggtatggatcgtaaaattttaagcactaaaaattttttcataacctttcataataaagtttaataataggtttattaactgaatttcattagttttttaaaagtgtttttggtttgtgtatatatacatatacaaatacaacatttacaataaataaaatacttgaaattctcttttgtgtctcctagtagcttcctactcaactatttataatctcattaattaaaaagttataattttagataaaaattctagtcaaatttttacagatattatctcactaattttcagacttttgccaaagtgtgcacaatggctttttgttaataaagaacagattagttttgaagaaggcaaaaatttcagttttctgaagacagcatgttattttaacaatcaagtatacatattaaaaattgtgagcaatctcaaaaaaaaaaaaaaa

SEQ ID NO: 5 - DNA, 4111 bp, Homo sapiens
cggcagggttggaaaatgatggaagaggcggaggtggaggcgaccgagtgctgagaggaacctgcggaatcggccgagatggggtctggcgcgcgctttccctcggggacccttcgtgtccggtggttgctgttgcttggcctggtgggcccagtcctcggtgcggcgcggccaggctttcaacagacctcacatctttcttcttatgaaattataactccttggagattaactagagaaagaagagaagcccctaggccctattcaaaacaagtatcttatgttattcaggctgaaggaaaagagcatattattcacttggaaaggaacaaagaccttttgcctgaagattttgtggtttatacttacaacaaggaagggactttaatcactgaccatcccaatatacagaatcattgtcattatcggggctatgtggagggagttcataattcatccattgctcttagcgactgttttggactcagaggattgctgcatttagagaatgcgagttatgggattgaacccctgcagaacagctctcattttgagcacatcatttatcgaatggatgatgtctacaaagagcctctgaaatgtggagtttccaacaaggatatagagaaagaaactgcaaaggatgaagaggaagagcctcccagcatgactcagctacttcgaagaagaagagctgtcttgccacagacccggtatgtggagctgttcattgtcgtagacaaggaaaggtatgacatgatgggaagaaatcagactgctgtgagagaagagatgattctcctggcaaactacttggatagtatgtatattatgttaaatattcgaattgtgctagttggactggagatttggaccaatggaaacctgatcaacatagttgggggtgctggtgatgtgctggggaacttcgtgcagtggcgggaaaagtttcttatcacacgtcggagacatgacagtgcacagctagttctaaagaaaggttttggtggaactgcaggaatggcatttgtgggaacagtgtgttcaaggagccacgcaggcgggattaatgtgtttggacaaatcactgtggagacatttgcttccattgttgctcatgaattgggtcataatcttggaatgaatcacgatgatgggagagattgttcctgtggagcaaagagctgcatcatgaattcaggagcatcgggttccagaaactttagcagttgcagtgcagaggactttgagaagttaactttaaataaaggaggaaactgccttcttaatattccaaagcctgatgaagcctatagtgctccctcctgtggtaataagttggtggacgctggggaagagtgtgactgtggtactccaaaggaatgtgaattggacccttgctgcgaaggaagtacctgtaagcttaaatcatttgctgagtgtgcatatggtgactgttgtaaagactgtcggttccttccaggaggtactttatgccgaggaaaaaccagtgagtgtgatgttccagagtactgcaatggttcttctcagttctgtcagccagatgtttttattcagaatggatatccttgccagaataacaaagcctattgctacaacggcatgtgccagtattatgatgctcaatgtcaagtcatctttggctcaaaagccaaggctgcccccaaagattgtttcattgaagtgaattctaaaggtgacagatttggcaattgtggtttctctggcaatgaatacaagaagtgtgccactgggaatgctttgtgtggaaagcttcagtgtgagaatgtacaagagatacctgtatttggaattgtgcctgctattattcaaacgcctagtcgaggcaccaaatgttggggtgtggatttccagctaggatcagatgttccagatcctgggatggttaacgaaggcacaaaatgtggtgctggaaagatctgtagaaacttccagtgtgtagatgcttctgttctgaattatgactgtgatgttcagaaaaagtgtcatggacatggggtatgtaatagcaataagaattgtcactgtgaaaatggctgggctcccccaaattgtgagactaaaggatacggaggaagtgtggacagtggacctacatacaatgaaatgaatactgcattgagggacggacttctggtcttcttcttcctaattgttccccttattgtctgtgctatttttatcttcatcaagagggatcaactgtggagaagctacttcagaaagaagagatcacaaacatatgagtcagatggcaaaaatcaagcaaacccttctagacagccggggagtgttcctcgacatgtttctccagtgacacctcccagagaagttcctatatatgcaaacagatttgcagtaccaacctatgcagccaagcaacctcagcagttcccatcaaggccacctccaccacaaccgaaagtatcatctcagggaaacttaattcctgcccgtcctgctcctgcacctcctttatatagttccctcacttgatttttttaaccttctttttgcaaatgtcttcagggaactgagctaatactttttttttttcttgatgttttcttgaaaagcctttctgttgcaactatgaatgaaaacaaaacaccacaaaacagacttcactaacacagaaaaacagaaactgagtgtgagagttgtgaaatacaaggaaatgcagtaaagccagggaatttacaataacatttccgtttccatcattgaataagtcttattcagtcatcggtgaggttaatgcactaatcatggattttttgaacatgttattgcagtgattctcaaattaactgtattggtgtaagatttttgtcattaagtgtttaagtgttattctgaattttctaccttagttatcattaatgtagttcctcattgaacatgtgataatctaatacctgtgaaaactgactaatcagctgccaataatatctaatatttttcatcatgcacgaattaataatcatcatactctagaatcttgtctgtcactcactacatgaataagcaaatattgtcttcaaaagaatgcacaagaaccacaattaagatgtcatattattttgaaagtacaaaatatactaaaagagtgtgtgtgtattcacgcagttactcgcttccatttttatgacctttcaactataggtaataactcttagagaaattaatttaatattagaatttctattatgaatcatgtgaaagcatgacattcgttcacaatagcactattttaaataaattataagctttaaggtacgaagtatttaatagatctaatcaaatatgttgattcatggctataataaagcaggagcaattataaaatcttcaatcaattgaacttttacaaaaccacttgagaatttcatgagcactttaaaatctgaactttcaaagcttgctattaaatcatttagaatgtttacatttactaaggtgtgctgggtcatgtaaaatattagacactaatattttcatagaaattaggctggagaaagaaggaagaaatggttttcttaaatacctacaaaaaagttactgtggtatctatgagttatcatcttagctgtgttaaaaatgaatttttactatggcagatatggtatggatcgtaaaattttaagcactaaaaattttttcataacctttcataataaagtttaataataggtttattaactgaatttcattagttttttaaaagtgtttttggtttgtgtatatatacatatacaaatacaacatttacaataaataaaatacttgaaattctcttttgtgtctcctagtagcttcctactcaactatttataatctcattaattaaaaagttataattttagataaaaattctagtcaaatttttacagatattatctcactaattttcagacttttgccaaagtgtgcacaatggctttttgttaataaagaacagattagttttgaagaaggcaaaaatttcagttttctgaagacagcatgttattttaacaatcaagtatacatattaaaaattgtgagcaatctcaaaaaaaaaaaaaaa

SEQ ID NO: 6 - DNA, 28 bp, Artificial Sequence
  Description of Artificial Sequence Synthetic primer
gatgctctttggaaactgaatggcacca

SEQ ID NO: 7 - DNA, 29 bp, Artificial Sequence
  Description of Artificial Sequence Synthetic primer
agtcgctgtcttgtcaattccaaaagcgt

SEQ ID NO: 8 - DNA, 24 bp, Artificial Sequence
  Description of Artificial Sequence Synthetic primer
atgacgtatgatgaagccagtgca

SEQ ID NO: 9 - DNA, 24 bp, Artificial Sequence
  Description of Artificial Sequence Synthetic primer
gttcctgattgttttgaacctaga

SEQ ID NO: 10 - DNA, 22 bp, Artificial Sequence
  Description of Artificial Sequence Synthetic primer
atgagtcttgacaacaacggta

SEQ ID NO: 11 - DNA, 25 bp, Artificial Sequence
  Description of Artificial Sequence Synthetic primer
tcacaattcggtatcagccaccacg

SEQ ID NO: 12 - DNA, 23 bp, Artificial Sequence
  Description of Artificial Sequence Synthetic primer
actgcatgcagagaaaaacagta

SEQ ID NO: 13 - DNA, 21 bp, Artificial Sequence
  Description of Artificial Sequence Synthetic primer
tcactgtctctcctgcactga

SEQ ID NO: 14 - DNA, 24 bp, Artificial Sequence
  Description of Artificial Sequence Synthetic primer
aatgcccagacatctgtgtccccc

SEQ ID NO: 15 - DNA, 20 bp, Artificial Sequence
  Description of Artificial Sequence Synthetic primer
tcagggaggcgtggcttgtg

SEQ ID NO: 16 - DNA, 19 bp, Artificial Sequence
  Description of Artificial Sequence Synthetic primer
gagatctcccctgaataca

SEQ ID NO: 17 - DNA, 21 bp, Artificial Sequence
  Description of Artificial Sequence Synthetic primer
ctacactttggatttctgtgc

SEQ ID NO: 18 - DNA, 23 bp, Artificial Sequence
  Description of Artificial Sequence Synthetic primer
tccctgcaaacaacaagtgatca

SEQ ID NO: 19 - DNA, 22 bp, Artificial Sequence
  Description of Artificial Sequence Synthetic primer
aaagttgccctctccgatcacg

SEQ ID NO: 20 - DNA, 22 bp, Artificial Sequence
  Description of Artificial Sequence Synthetic primer
agggttggaaaatgatggaaga

SEQ ID NO: 21 - DNA, 20 bp, Artificial Sequence
  Description of Artificial Sequence Synthetic primer
gattccgcaggttcctctca

SEQ ID NO: 22 - DNA, 21 bp, Artificial Sequence
  Description of Artificial Sequence Synthetic probe
cggaggtggaggcgaccgagt

SEQ ID NO: 23 - DNA, 20 bp, Artificial Sequence
  Description of Artificial Sequence Synthetic oligonucleotide
cctcttcatcctttgcagtt

SEQ ID NO: 24 - DNA, 20 bp, Artificial Sequence
  Description of Artificial Sequence Synthetic oligonucleotide
gtttccattggtccaaatct

SEQ ID NO: 25 - DNA, 20 bp, Artificial Sequence
  Description of Artificial Sequence Synthetic oligonucleotide
caagattatgacccaattca

SEQ ID NO: 26 - DNA, 20 bp, Artificial Sequence
  Description of Artificial Sequence Synthetic oligonucleotide
gaatattaagaaggcagttt

SEQ ID NO: 27 - DNA, 20 bp, Artificial Sequence
  Description of Artificial Sequence Synthetic oligonucleotide
ctgctttattatagccatga

SEQ ID NO: 28 - DNA, 20 bp, Artificial Sequence
  Description of Artificial Sequence Synthetic oligonucleotide
atgactgaataagacttatt

SEQ ID NO: 29 - DNA, 20 bp, Artificial Sequence
  Description of Artificial Sequence Synthetic oligonucleotide
tacattaatgataactaagg

SEQ ID NO: 30 - DNA, 20 bp, Artificial Sequence
  Description of Artificial Sequence Synthetic oligonucleotide
aagattttataattgctcct

SEQ ID NO: 31 - DNA, 20 bp, Artificial Sequence
  Description of Artificial Sequence Synthetic oligonucleotide
cgataaatgatgtgctcaaa

SEQ ID NO: 32 - DNA, 20 bp, Artificial Sequence
  Description of Artificial Sequence Synthetic oligonucleotide
cagtgatttgtccaaacaca

SEQ ID NO: 33 - DNA, 20 bp, Artificial Sequence
  Description of Artificial Sequence Synthetic oligonucleotide
attcattccaagattatgac

SEQ ID NO: 34 - DNA, 20 bp, Artificial Sequence
  Description of Artificial Sequence Synthetic oligonucleotide
ttaatagcaagctttgaaag

SEQ ID NO: 35 - DNA, 20 bp, Artificial Sequence
  Description of Artificial Sequence Synthetic oligonucleotide
gggtctgtggcaagacagct

SEQ ID NO: 36 - DNA, 20 bp, Artificial Sequence
  Description of Artificial Sequence Synthetic oligonucleotide
tctttgctccacaggaacaa

SEQ ID NO: 37 - DNA, 20 bp, Artificial Sequence
  Description of Artificial Sequence Synthetic oligonucleotide
caatggatgaattatgaact

SEQ ID NO: 38 - DNA, 20 bp, Artificial Sequence
  Description of Artificial Sequence Synthetic oligonucleotide
tccgtatcctttagtctcac

SEQ ID NO: 39 - DNA, 20 bp, Artificial Sequence
  Description of Artificial Sequence Synthetic oligonucleotide
ttccttctttctccagccta

SEQ ID NO: 40 - DNA, 20 bp, Artificial Sequence
  Description of Artificial Sequence Synthetic oligonucleotide
gtgagggaactatataaagg

SEQ ID NO: 41 - DNA, 20 bp, Artificial Sequence
  Description of Artificial Sequence Synthetic oligonucleotide
aggaattaagtttccctgag

SEQ ID NO: 42 - DNA, 20 bp, Artificial Sequence
  Description of Artificial Sequence Synthetic oligonucleotide
aaaattttacgatccatacc

SEQ ID NO: 43 - DNA, 20 bp, Artificial Sequence
  Description of Artificial Sequence Synthetic oligonucleotide
ctggaacatctgatcctagc

SEQ ID NO: 44 - DNA, 20 bp, Artificial Sequence
  Description of Artificial Sequence Synthetic oligonucleotide
caaatgtctccacagtgatt

SEQ ID NO: 45 - DNA, 20 bp, Artificial Sequence
  Description of Artificial Sequence Synthetic oligonucleotide
gctattgtgaacgaatgtca

SEQ ID NO: 46 - DNA, 20 bp, Artificial Sequence
  Description of Artificial Sequence Synthetic oligonucleotide
caagattctagagtatgatg

SEQ ID NO: 47 - DNA, 20 bp, Artificial Sequence
  Description of Artificial Sequence Synthetic oligonucleotide
atcatgtcatacctttcctt

SEQ ID NO: 48 - DNA, 20 bp, Artificial Sequence
  Description of Artificial Sequence Synthetic oligonucleotide
gggaggctcttcctcttcat

SEQ ID NO: 49 - DNA, 20 bp, Artificial Sequence
  Description of Artificial Sequence Synthetic oligonucleotide
actgaagctttccacacaaa

SEQ ID NO: 50 - DNA, 20 bp, Artificial Sequence
  Description of Artificial Sequence Synthetic oligonucleotide
tcccacaaatgccattcctg

SEQ ID NO: 51 - DNA, 20 bp, Artificial Sequence
  Description of Artificial Sequence Synthetic oligonucleotide
agaaaaccatttcttccttc

SEQ ID NO: 52 - DNA, 20 bp, Artificial Sequence
  Description of Artificial Sequence Synthetic oligonucleotide
attattggcagctgattagt

SEQ ID NO: 53 - DNA, 20 bp, Artificial Sequence
  Description of Artificial Sequence Synthetic oligonucleotide
gcaccacattttgtgccttc

SEQ ID NO: 54 - DNA, 20 bp, Artificial Sequence
  Description of Artificial Sequence Synthetic oligonucleotide
ctgattagtcagttttcaca

SEQ ID NO: 55 - DNA, 20 bp, Artificial Sequence
  Description of Artificial Sequence Synthetic oligonucleotide
tcaatgcagtattcatttca

SEQ ID NO: 56 - DNA, 20 bp, Artificial Sequence
  Description of Artificial Sequence Synthetic oligonucleotide
aagaagaccagaagtccgtc

SEQ ID NO: 57 - DNA, 20 bp, Artificial Sequence
  Description of Artificial Sequence Synthetic oligonucleotide
attcctttggagtaccacag

SEQ ID NO: 58 - DNA, 20 bp, Artificial Sequence
  Description of Artificial Sequence Synthetic oligonucleotide
ggtatctcttgtacattctc

SEQ ID NO: 59 - DNA, 20 bp, Artificial Sequence
  Description of Artificial Sequence Synthetic oligonucleotide
gggaactgctgaggttgctt

SEQ ID NO: 60 - DNA, 20 bp, Homo sapiens
aactgcaaaggatgaagagg

SEQ ID NO: 61 - DNA, 20 bp, Homo sapiens
agatttggaccaatggaaac

SEQ ID NO: 62 - DNA, 20 bp, Homo sapiens
tgaattgggtcataatcttg

SEQ ID NO: 63 - DNA, 20 bp, Homo sapiens
tcatggctataataaagcag

SEQ ID NO: 64 - DNA, 20 bp, Homo sapiens
aataagtcttattcagtcat

SEQ ID NO: 65 - DNA, 20 bp, Homo sapiens
ccttagttatcattaatgta

SEQ ID NO: 66 - DNA, 20 bp, Homo sapiens
aggagcaattataaaatctt

SEQ ID NO: 67 - DNA, 20 bp, Homo sapiens
gtcataatcttggaatgaat

SEQ ID NO: 68 - DNA, 20 bp, Homo sapiens
ctttcaaagcttgctattaa

SEQ ID NO: 69 - DNA, 20 bp, Homo sapiens
agctgtcttgccacagaccc

SEQ ID NO: 70 - DNA, 20 bp, Homo sapiens
ttgttcctgtggagcaaaga

SEQ ID NO: 71 - DNA, 20 bp, Homo sapiens
agttcataattcatccattg

SEQ ID NO: 72 - DNA, 20 bp, Homo sapiens
gtgagactaaaggatacgga

SEQ ID NO: 73 - DNA, 20 bp, Homo sapiens
taggctggagaaagaaggaa

SEQ ID NO: 74 - DNA, 20 bp, Homo sapiens
cctttatatagttccctcac

SEQ ID NO: 75 - DNA, 20 bp, Homo sapiens
ctcagggaaacttaattcct

SEQ ID NO: 76 - DNA, 20 bp, Homo sapiens
ggtatggatcgtaaaatttt

SEQ ID NO: 77 - DNA, 20 bp, Homo sapiens
gctaggatcagatgttccag

SEQ ID NO: 78 - DNA, 20 bp, Homo sapiens
aatcactgtggagacatttg

SEQ ID NO: 79 - DNA, 20 bp, Homo sapiens
tgacattcgttcacaatagc

SEQ ID NO: 80 - DNA, 20 bp, Homo sapiens
catcatactctagaatcttg

SEQ ID NO: 81 - DNA, 20 bp, Homo sapiens
aaggaaaggtatgacatgat

SEQ ID NO: 82 - DNA, 20 bp, Homo sapiens
atgaagaggaagagcctccc

SEQ ID NO: 83 - DNA, 20 bp, Homo sapiens
tttgtgtggaaagcttcagt

SEQ ID NO: 84 - DNA, 20 bp, Homo sapiens
caggaatggcatttgtggga

SEQ ID NO: 85 - DNA, 20 bp, Homo sapiens
gaaggaagaaatggttttct

SEQ ID NO: 86 - DNA, 20 bp, Homo sapiens
actaatcagctgccaataat

SEQ ID NO: 87 - DNA, 20 bp, Homo sapiens
gaaggcacaaaatgtggtgc

SEQ ID NO: 88 - DNA, 20 bp, Homo sapiens
tgtgaaaactgactaatcag

SEQ ID NO: 89 - DNA, 20 bp, Homo sapiens
tgaaatgaatactgcattga

SEQ ID NO: 90 - DNA, 20 bp, Homo sapiens
gacggacttctggtcttctt

SEQ ID NO: 91 - DNA, 20 bp, Homo sapiens
ctgtggtactccaaaggaat

SEQ ID NO: 92 - DNA, 20 bp, Homo sapiens
aagcaacctcagcagttccc

SEQ ID NO: 93 - protein, 12 aa, Homo sapiens
HELGHNLGMNHD

SEQ ID NO: 94 - DNA, 3865 bp, Homo sapiens
  CDS (79)..(2535)
cggcagggttggaaaatgatggaagaggcggaggtggaggcgaccgagtgctgagaggaacctgcggaatcggccgagatggggtctggcgcgcgctttccctcggggacccttcgtgtccggtggttgctgttgcttggcctggtgggcccagtcctcggtgcggcgcggccaggctttcaacagacctcacatctttcttcttatgaaattataactccttggagattaactagagaaagaagagaagcccctaggccctattcaaaacaagtatcttatgttattcaggctgaaggaaaagagcatattattcacttggaaaggaacaaagaccttttgcctgaagattttgtggtttatacttacaacaaggaagggactttaatcactgaccatcccaatatacagaatcattgtcattatcggggctatgtggagggagttcataattcatccattgctcttagcgactgttttggactcagaggattgctgcatttagagaatgcgagttatgggattgaacccctgcagaacagctctcattttgagcacatcatttatcgaatggatgatgtctacaaagagcctctgaaatgtggagtttccaacaaggatatagagaaagaaactgcaaaggatgaagaggaagagcctcccagcatgactcagctacttcgaagaagaagagctgtcttgccacagacccggtatgtggagctgttcattgtcgtagacaaggaaaggtatgacatgatgggaagaaatcagactgctgtgagagaagagatgattctcctggcaaactacttggatagtatgtatattatgttaaatattcgaattgtgctagttggactggagatttggaccaatggaaacctgatcaacatagttgggggtgctggtgatgtgctggggaacttcgtgcagtggcgggaaaagtttcttatcacacgtcggagacatgacagtgcacagctagttctaaagaaaggttttggtggaactgcaggaatggcatttgtgggaacagtgtgttcaaggagccacgcaggcgggattaatgtgtttggacaaatcactgtggagacatttgcttccattgttgctcatgaattgggtcataatcttggaatgaatcacgatgatgggagagattgttcctgtggagcaaagagctgcatcatgaattcaggagcatcgggttccagaaactttagcagttgcagtgcagaggactttgagaagttaactttaaataaaggaggaaactgccttcttaatattccaaagcctgatgaagcctatagtgctccctcctgtggtaataagttggtggacgctggggaagagtgtgactgtggtactccaaaggaatgtgaattggacccttgctgcgaaggaagtacctgtaagcttaaatcatttgctgagtgtgcatatggtgactgttgtaaagactgtcggttccttccaggaggtactttatgccgaggaaaaaccagtgagtgtgatgttccagagtactgcaatggttcttctcagttctgtcagccagatgtttttattcagaatggatatccttgccagaataacaaagcctattgctacaacggcatgtgccagtattatgatgctcaatgtcaagtcatctttggctcaaaagccaaggctgcccccaaagattgtttcattgaagtgaattctaaaggtgacagatttggcaattgtggtttctctggcaatgaatacaagaagtgtgccactgggaatgctttgtgtggaaagcttcagtgtgagaatgtacaagagatacctgtatttggaattgtgcctgctattattcaaacgcctagtcgaggcaccaaatgttggggtgtggatttccagctaggatcagatgttccagatcctgggatggttaacgaaggcacaaaatgtggtgctggaaagatctgtagaaacttccagtgtgtagatgcttctgttctgaattatgactgtgatgttcagaaaaagtgtcatggacatggggtatgtaatagcaataagaattgtcactgtgaaaatggctgggctcccccaaattgtgagactaaaggatacggaggaagtgtggacagtggacctacatacaatgaaatgaatactgcattgagggacggacttctggtcttcttcttcctaattgttccccttattgtctgtgctatttttatcttcatcaagagggatcaactgtggagaagctacttcagaaagaagagatcacaaacatatgagtcagatggcaaaaatcaagcaaacccttctagacagccggggagtgttcctcgacatgtttctccagtgacacctcccagagaagttcctatatatgcaaacagatttgcagtaccaacctatgcagccaagcaacctcagcagttcccatcaaggccacctccaccacaaccgaaagtatcatctcagggaaacttaattcctgcccgtcctgctcctgcacctcctttatatagttccctcacttgatttttttaaccttctttttgcaaatgtcttcagggaactgagctaatactttttttttttcttgatgttttcttgaaaagcctttctgttgcaactatgaatgaaaacaaaacaccacaaaacagacttcactaacacagaaaaacagaaactgagtgtgagagttgtgaaatacaaggaaatgcagtaaagccagggaatttacaataacatttccgtttccatcattgaataagtcttattcagtcatcggtgaggttaatgcactaatcatggattttttgaacatgttattgcagtgattctcaaattaactgtattggtgtaagatttttgtcattaagtgtttaagtgttattctgaattttctaccttagttatcattaatgtagttcctcattgaacatgtgataatctaatacctgtgaaaactgactaatcagctgccaataatatctaatatttttcatcatgcacgaattaataatcatcatactctagaatcttgtctgtcactcactacatgaataagcaaatattgtcttcaaaagaatgcacaagaaccacaattaagatgtcatattattttgaaagtacaaaatatactaaaagagtgtgtgtgtattcacgcagttactcgcttccatttttatgacctttcaactataggtaataactcttagagaaattaatttaatattagaatttctattatgaatcatgtgaaagcatgacattcgttcacaatagcactattttaaataaattataagctttaaggtacgaagtatttaatagatctaatcaaatatgttgattcatggctataataaagcaggagcaattataaaatcttcaatcaattgaacttttacaaaaccacttgagaatttcatgagcactttaaaatctgaactttcaaagcttgctattaaatcatttagaatgtttacatttactaaggtgtgctgggtcatgtaaaatattagacactaatattttcatagaaattaggctggagaaagaaggaagaaatggttttcttaaatacctacaaaaaagttactgtggtatctatgagttatcatcttagctgtgttaaaaatgaatttttactatggcagatatggtatggatcgtaaaattttaagcactaaaaattttttcataacctttcataataaagtttaataataggtttattaactgaatttcattagttttttaaaagtgtttttggtttgtgtatatatacatatacaaatacaacatttacaataaataaaatacttgaaattctcaaaaaaaaaaaaaaaaaaaaaaaaaa

SEQ ID NO: 95 - protein, 6 aa, Artificial Sequence
  Description of Artificial Sequence Synthetic 6xHis tag
HHHHHH